(19) United States
(12) Patent Application Publication
(10) Pub. No.: US 2004/0073155 A1
(43) Pub. Date: Apr. 15, 2004

(54) Methods and devices for maintaining patency of surgically created channels in tissue

(75) Inventors: Michael D. Laufer, Menlo Park, CA (US); Cary Cole, Mountain View, CA (US); Bryan Loomas, Los Gatos, CA (US); Gary Kaplan, San Francisco, CA (US); Cary J. Reich, Los Gatos, CA (US); Ed Roschak, Mission Viejo, CA (US); Trudy Estridge, Fremont, CA (US)

Correspondence Address: BRONCUS TECHNOLOGIES, INC., BUILDING A8, 1400 N. SHORELINE BLVD., MOUNTAIN VIEW, CA, 94043, US

(73) Assignee: Broncus Technologies, Inc., Mountain View, CA (US)

(21) Appl. No.: 10/458,085

(22) Filed: Jun. 9, 2003

Related U.S. Application Data
(63) Continuation-in-part of application No. 10/235,240, filed on Sep. 4, 2002.
Application No. 10/235,240 is a continuation-in-part of application No. 09/947,144, filed on Sep. 4, 2001.
Application No. 09/947,144 is a continuation-in-part of application No. 09/908,177, filed on Jul. 18, 2001.
Application No. 09/908,177 is a continuation of application No. 09/633,651, filed on Aug. 7, 2000, now Pat. No. 6,692,494.
(60) Provisional application No. 60/420,440, filed on Oct. 21, 2002.
(60) Provisional application No. 60/387,163, filed on Jun. 7, 2002.
(60) Provisional application No. 60/176,141, filed on Jan. 14, 2000.

(51) Int. Cl. A61F 2/04; A61B 19/00

(52) U.S. Cl. 604/8; 128/898; 623/23.65

(57) ABSTRACT

Devices and methods are directed to altering gaseous flow within a lung to improve the expiration cycle of, for instance, an individual having chronic obstructive pulmonary disease. More particularly, methods and devices are disclosed that inhibit closure of channels surgically created through an airway wall such that air is able to pass directly out of the lung tissue to facilitate both the exchange of oxygen ultimately into the blood and/or to decompress hyper-inflated lungs. Devices, instruments, medicine, bioactive agents, or combinations thereof serve to maintain the patency of the surgically created channels. In one embodiment of the present invention, a conduit includes a bioactive coating that inhibits tissue overgrowth when the conduit is deployed in a surgically created channel. Still other methods and devices are described that serve to maintain surgically created channels.

### FIELD OF THE INVENTION

This is directed to methods and devices for altering gaseous flow within a lung to improve the expiration cycle of an individual, particularly individuals having chronic obstructive pulmonary disease. The methods and devices maintain the patency of surgically created channels or openings in tissue. Maintaining the patency of the channels allows air to pass directly out of the lung tissue which facilitates the exchange of oxygen ultimately into the blood and/or decompresses hyper-inflated lungs.

### BACKGROUND OF THE INVENTION

The American Lung Association (ALA) estimates that nearly 16 million Americans suffer from chronic obstructive pulmonary disease (COPD) which includes diseases such as chronic bronchitis, emphysema, and some types of asthma. The ALA estimated that COPD was the fourth-ranking cause of death in the U.S. The ALA estimates that about 14 million and 2 million Americans suffer from emphysema and chronic bronchitis respectively.

Those inflicted with COPD face disabilities due to the limited pulmonary functions. Usually, individuals afflicted by COPD also face loss in muscle strength and an inability to perform common daily activities. Often, those patients desiring treatment for COPD seek a physician at a point where the disease is advanced. Since the damage to the lungs is irreversible, there is little hope of recovery. Most times, the physician cannot reverse the effects of the disease but can only offer treatment and advice to halt the progression of the disease.

To understand the detrimental effects of COPD, the workings of the lungs requires a cursory discussion. The primary function of the lungs is to permit the exchange of two gasses by removing carbon dioxide from arterial blood and replacing it with oxygen. Thus, to facilitate this exchange, the lungs provide a blood gas interface. The oxygen and carbon dioxide move between the gas (air) and blood by diffusion. This diffusion is possible since the blood is delivered to one side of the blood-gas interface via small blood vessels (capillaries). The capillaries are wrapped around numerous air sacs called alveoli which function as the blood-gas interface. A typical human lung contains about 300 million alveoli.

The air is brought to the other side of this blood-gas interface by a natural respiratory airway, hereafter referred to as a natural airway or airway, consisting of branching tubes which become narrower, shorter, and more numerous as they penetrate deeper into the lung. Specifically, the airway begins with the trachea which branches into the left and right bronchi which divide into lobar, then segmental bronchi. Ultimately, the branching continues down to the terminal bronchioles which lead to the alveoli. Plates of cartilage may be found as part of the walls throughout most of the airway from the trachea to the bronchi. The cartilage plates become less prevalent as the airways branch. Eventually, in the last generations of the bronchi, the cartilage plates are found only at the branching points. The bronchi and bronchioles may be distinguished as the bronchi lie proximal to the last plate of cartilage found along the airway, while the bronchiole lies distal to the last plate of cartilage. The bronchioles are the smallest airways that do not contain alveoli. The function of the bronchi and bronchioles is to provide conducting airways that lead air to and from the gas-blood interface. However, these conducting airways do not take part in gas exchange because they do not contain alveoli. Rather, the gas exchange takes place in the alveoli which are found in the distal most end of the airways.

The mechanics of breathing include the lungs, the rib cage, the diaphragm and abdominal wall. During inspiration, inspiratory muscles contract increasing the volume of the chest cavity. As a result of the expansion of the chest cavity, the pleural pressure, the pressure within the chest cavity, becomes sub-atmospheric. Consequently, air flows into the lungs and the lungs expand. During unforced expiration, the inspiratory muscles relax and the lungs begin to recoil and reduce in size. The lungs recoil because they contain elastic fibers that allow for expansion, as the lungs inflate, and relaxation, as the lungs deflate, with each breath. This characteristic is called elastic recoil. The recoil of the lungs causes alveolar pressure to exceed atmospheric pressure causing air to flow out of the lungs and deflate the lungs. ‘If the lungs’ ability to recoil is damaged, the lungs cannot contract and reduce in size from their inflated state. As a result, the lungs cannot evacuate all of the inspired air.

In addition to elastic recoil, the lung's elastic fibers also assist in keeping small airways open during the exhalation cycle. This effect is also known as “tethering” of the airways. Tethering is desirable since small airways do not contain cartilage that would otherwise provide structural rigidity for these airways. Without tethering, and in the absence of structural rigidity, the small airways collapse during exhalation and prevent air from exiting thereby trapping air within the lung.

Emphysema is characterized by irreversible biochemical destruction of the alveolar walls that contain the elastic fibers, called elastin, described above. The destruction of the alveolar walls results in a dual problem of reduction of elastic recoil and the loss of tethering of the airways. Unfortunately for the individual suffering from emphysema, these two problems combine to result in extreme hyperinflation (air trapping) of the lung and an inability of the person to exhale. In this situation, the individual will be debilitated since the lungs are unable to perform gas exchange at a satisfactory rate.

One further aspect of alveolar wall destruction is that the airflow between neighboring air sacs, known as collateral ventilation or collateral air flow, is markedly increased as when compared to a healthy lung. While alveolar wall destruction decreases resistance to collateral ventilation, the resulting increased collateral ventilation does not benefit the individual since air is still unable to flow into and out of the lungs. Hence, because this trapped air is rich in CO₂, it is of little or no benefit to the individual.

Chronic bronchitis is characterized by excessive mucus production in the bronchial tree. Usually there is a general increase in bulk (hypertrophy) of the large bronchi and chronic inflammatory changes in the small airways. Excessive amounts of mucus are found in the airways and semisolid plugs of this mucus may occlude some small bronchi. Also, the small airways are usually narrowed and show inflammatory changes.

Currently, although there is no cure for COPD, treatment includes bronchodilator drugs, and lung reduction surgery. The bronchodilator drugs relax and widen the air passages thereby reducing the residual volume and increasing gas flow permitting more oxygen to enter the lungs. Yet, bronchodilator drugs are only effective for a short period of time and require repeated application. Moreover, the bronchodilator drugs are only effective in a certain percentage of the population of those diagnosed with COPD. In some cases, patients suffering from COPD are given supplemental oxygen to assist in breathing. Unfortunately, aside from the impracticalities of needing to maintain and transport a source of oxygen for everyday activities, the oxygen is only partially functional and does not eliminate the effects of the COPD. Moreover, patients requiring a supplemental source of oxygen are usually never able to return to functioning without the oxygen.

Lung volume reduction surgery is a procedure which removes portions of the lung that are over-inflated. The portion of the lung that remains has relatively better elastic recoil, providing reduced airway obstruction. The reduced lung volume also improves the efficiency of the respiratory muscles. However, lung reduction surgery is an extremely traumatic procedure which involves opening the chest and thoracic cavity to remove a portion of the lung. As such, the procedure involves an extended recovery period. Hence, the long term benefits of this surgery are still being evaluated. In any case, it is thought that lung reduction surgery is sought in those cases of emphysema where only a portion of the lung is emphysematous as opposed to the case where the entire lung is emphysematous. In cases where the lung is only partially emphysematous, removal of a portion of emphysematous lung which was compressing healthier portions of the lung allows the healthier portions to expand, increasing the overall efficiency of the lung. If the entire lung is emphysematous, however, removal of a portion of the lung removes gas exchanging alveolar surfaces, reducing the overall efficiency of the lung. Lung volume reduction surgery is thus not a practical solution for treatment of emphysema where the entire lung is diseased.

Both bronchodilator drugs and lung reduction surgery fail to capitalize on the increased collateral ventilation taking place in the diseased lung. There remains a need for a medical procedure that can alleviate some of the problems caused by COPD. There is also a need for a medical procedure that alleviates some of the problems caused by COPD irrespective of whether a portion of the lung, or the entire lung is emphysematous. The production and maintenance of collateral openings through an airway wall allows air to pass directly out of the lung tissue responsible for gas exchange. These collateral openings serve to decompress hyper inflated lungs and/or facilitate an exchange of oxygen into the blood.

Methods and devices for creating and maintaining collateral channels are discussed in U.S. Patent Application Ser. No. 09/633,651, filed on Aug. 7, 2000; U.S. patent application Ser. Nos. 09/947,144, 09/946,706, and 09/947,126 all filed on Sep. 4, 2001; U.S. Provisional Application No. 60/317,338 filed on Sep. 4, 2001; U.S. Provisional Application No. 60/334,642 filed on Nov. 29, 2001; U.S. Provisional Application No. 60/367,436 filed on Mar. 20, 2002; and U.S. Provisional Application No. 60/374,022 filed on Apr. 19, 2002 each of which is incorporated by reference herein in its entirety.

Although creating an opening through an airway wall may overcome the shortcomings associated with bronchodilator drugs and lung volume reduction surgery, various problems can still arise. When a hole is surgically created in tissue the healing cascade is triggered. The body's natural healing responses are set into motion including, amongst other things, cell proliferation which can result in a build-up of scar tissue. This tissue overgrowth can occlude or otherwise close the surgically created opening. Additionally, in the event an implant is deployed in the surgically created opening to maintain the patency of the opening, the implant may become encapsulated or filled with tissue thereby occluding the channel.

Drug eluting coronary-type stents are not known to overcome the above mentioned events because the stents are often substantially cylindrical (or otherwise have a shape that conforms to the shape of a tubular blood vessel). Hence, they may slide and be ejected from surgically created openings in an airway wall. Additionally, the drugs eluted from these stents are generally intended to inhibit platelet, fibrin and thrombin aggregation/formation and perhaps, prevent proliferation of certain types of cells found in the blood vessels. The cells lining the internal surfaces of the blood vessels are simple squamous epithelium (endothelia) cells and are a derivative of the mesoderm, a primary germ layer during embryonic development.

In contrast, the internal lining of an airway comprises pseudostratified columnar epithelial cells and cuboidal epithelial cells corresponding to the upper respiratory tract (e.g., trachea and bronchi) and the bronchioles respectively. These epithelial cells are, amongst other things, shaped differently than the cells lining the blood vessels. These airway epithelial cells are a derivative tissue of the endoderm embryonic germ layer, a different embryonic germ layer than that corresponding to the blood vasculature.

There are other notable differences between the blood vessels and the airways. The airways are secretory in nature. The airway epithelia comprises mucous gland cells and, in the upper respiratory tract, cilia. The airways transport air and mucus. In contrast, the blood vessels transport blood. Blood contains, amongst other things, plaque, platelets, blood cells, fibrin, and thrombin.

Blood passing through the blood vessels may generate different shear forces on the inner surface of the vessel than that corresponding to air passing through the airways. Also, the epithelial cells of the airways are subject to bi-directional flow (of air during inhalation and exhalation) whereas the endothelial cells of the blood vessels are subject to uni-directional blood flow. Also, the temperature in the airway may be different than that found in a blood vessel. The temperature of an airway is closer to the temperature of the air outside the body. The temperature found in a blood vessel is at body temperature since the blood transports/transfers heat to the blood vessel wall.

Accordingly, devices and methods that specifically address the healing mechanisms of the airways are desired to provide long-term patency of surgically-created channels in the airways and in particular, to prevent tissue ingrowth from occluding the surgically-created channels.

### SUMMARY OF THE INVENTION

Devices and methods serve to maintain the patency of a channel surgically created in tissue such as an airway wall. In particular, the devices and methods prevent closure of the channel such that air may flow through the channel and into the airway. Such channels may be made by a variety of methods as discussed in the patents incorporated by reference above. For example, the channel may be made via a surgical incision, a needle, a rotary coring device, etc. Furthermore, the channel may be made by an energy based device, e.g., RF device, laser, etc. However, it has been noted that use of low temperature devices, e.g., mechanical devices, to create the channel result in less trauma to surrounding tissue and thereby minimize the healing response of the tissue. Accordingly, such modes of creating the channel often result in less occlusion of the channel.

Preventing closure may be performed using various approaches including, but not limited to, biochemical, electrical, thermal, irradiation, or mechanical approaches (or any combination thereof).

Biochemical approaches include delivery of medicines that inhibit closure of the surgically created channel. The medicines may be delivered locally or systematically. In one variation, a delivery catheter includes a dispense lumen that sends a drug to the target site. Also, bioactive substances may be delivered to the channel tissue using various delivery vehicles such as a conduit. The bioactive substance may be disposed on an exterior surface of the conduit such that it interacts with the channel tissue when the conduit is placed at the injury site. Also, bioactive substances may be delivered to the channel tissue before or after the conduit is positioned in the channel. The bioactive agent may also be delivered to the target site alone. That is, a medicine may be sent to the surgically created channel as the sole mechanism for maintaining the patency of the channel.

Also, systematic delivery of medicines may be carried out through digestion, injection, inhalation, etc. Systematic delivery of medicines may be provided alone or in combination with other techniques described herein.

Various bioactive substances may be used to prevent closure of the channel. The bioactive substances are intended 1.) to accelerate tissue growth (or healing) in the shape of a surgically-created channel or 2.) to inhibit or halt growth of the tissue such that patency of the channel is maintained. Examples of substances include but are not limited to antithrobotics, anticoagulants, antiplatelet agents, thorombolytics, antiproliferatives, antinflammatories, agents that inhibit hyperplasia and in particular restenosis, smooth muscle cell inhibitors, growth factors, growth factor inhibitors, cell adhesion inhibitors, cell adhesion promoters and drugs that may enhance the formation of healthy neointimal tissue, including endothelial cell regeneration. The positive action may come from inhibiting particular cells (e.g., smooth muscle cells) or tissue formation (e.g., fibromuscular tissue) while encouraging different cell migration (e.g., endothelium, epithelium) and tissue formation (neointimal tissue).

Still other bioactive agents in carrying out the present invention include but are not limited to analgesics, anticonvulsives, anti-infectives (e.g., antibiotics, antimicrobials), antineoplastics, H2 antagonists (Histamine 2 antagonists), steroids, non-steroidal anti-inflammatories, hormones, immunomodulators, mast cell stabilizers, nucleoside analogues, respiratory agents, antihypertensives, antihistamines, ACE inhibitors, cell growth factors, nerve growth factors, anti-angiogenic agents or angiogenesis inhibitors (e.g., endostatins or angiostatins), tissue irritants (e.g., a compound comprising talc), poisons (e.g., arsenic), cytotoxic agents (e.g., a compound that can cause cell death), metals (silver, aluminum, zinc, platinum, arsenic, etc.), or a combination of any of the agents disclosed herein.

Additionally, lung-active substances may be used to prevent closure including lung-active substances which affect the rate of wound healing in lung tissue. Examples of agents include but are not limited to: agents that affect the growth or production (e.g., facilitate or retard) of epithelial cuboidal cells, epithelial pseudostratified columnar cells, and other tissues derived from the mesoderm embryonic germ layer.

Examples of agents include but are not limited to: pyrolitic carbon, titanium-nitride-oxide, paclitaxel, fibrinogen, collagen, thrombin, phosphorylcholine, heparin, rapamycin, radioactive 188Re and 32P, silver nitrate, dactinomycin, sirolimus, everolimus, or cell adhesion peptides.

Tissue adhesives, cells, proteins, grafts and additional implants may also be used alone or in combination with the conduits and other mechanisms disclosed herein.

Light, thermal, electrical, ultrasonic energy may be delivered to the channel to prevent tissue build-up or otherwise prevent the channel from closing. An implant such as a conduit may be deployed in the channel in combination with any of the above mentioned approaches to prevent tissue build-up from occluding the channel.

Maintenance of the channel may also include clearing tissue build-up or overgrowth present in the channel. In the event a conduit or another type of implant is deployed in the channel, the maintenance of the conduit may include clearing the passage of the conduit. This maintenance may be performed one-time only, periodically, or responsive to the severity of the occlusion. The maintenance may be performed using light, laser, thermal, electrical, chemical (e.g., a reaction), and/or mechanical energy (e.g., cutting, scraping etc.).

Mechanical approaches for maintaining the patency of surgically created channels include, for example, deploying a conduit. The conduit may comprise a radially expandable center section having a first end and a second end and a passageway extending between the ends. The conduit may further include at least one center-control segment configured to restrict radial expansion of the passageway to a maximum profile. At least one extension member may extend from each of the first and second ends of the center section and each of the extension members may have a fixed end connected to one of the ends of the center section and a movable end such that each of the extension members is capable of being deflected about the fixed end.

The conduit may further be associated with a bioactive substance. The bioactive substance may be disposed on at least a portion of a surface of the conduit. The bioactive substance may serve to reduce tissue growth such that the conduit remains in the channel and the passageway remains at least partially open. The bioactive substance may be disposed on regions of the surface corresponding to the center section, the extension members, both the center section and extension members, or portions of these features.

The conduit may comprise a mesh formed from a plurality of ribs or strands. Additionally, the conduit may comprise a tissue barrier coaxially covering the passageway. The tissue barrier may form an exterior surface upon which the bioactive substance is disposed or the tissue barrier may be integral with or entirely composed of the bioactive substance. The tissue barrier may further cover at least a portion of the extension members or the entire lengths of the extension members.

In a variation, the bioactive substance is combined with a bioabsorbable polymer layer that gradually elutes when the conduit is deployed in a surgically-created channel.

The conduit may comprise at least one visualization feature disposed on a portion of the tissue barrier. The visualization feature may be a stripe circumferentially disposed about at least a portion of the center section. The visualization feature serves to aid in placement or deployment of the conduit in a target site.

Another conduit for maintaining the patency of a channel created in tissue comprises a radially expandable center section and extension members as described above. A bioactive substance is disposed on at least a portion of a surface of the conduit. Also, when the conduit is radially expanded it has an overall length and an inner diameter such that a ratio of the overall length to the inner diameter ranges from 1/6 to 2/1. The conduit may also be provided such that this ratio ranges from 1/4 to 1/1 and perhaps, 1/4 to 1/2. A tissue barrier may be disposed on at least a portion of the exterior surface corresponding to the center section. The tissue barrier may be comprised of various materials including but not limited to polymers and elastomers. An example of a material which may be used for the tissue barrier is silicone. Additional matrixes of biodegradable polymer and medicines may be associated with the tissue barrier such that controlled doses of medicines are delivered to the tissue opening.

This application is also related to the following applications No. 60/420,440 filed Oct. 21, 2002; No. 60/387,163 filed Jun. 7, 2002; Ser. No. 10/235,240 filed Sep. 4, 2002; Ser. No. 09/947,144 filed Sep. 4, 2001; Ser. No. 09/908,177 filed Jul. 18, 2001; Ser. No. 09/633,651 filed Aug. 7, 2000; and No. 60/176,141 filed Jan. 14, 2000; Ser. No. 10/080,344 filed Feb. 21, 2002; Ser. No. 10/079,605 filed Feb. 21, 2002; and Ser. No. 10/280,851 filed Oct. 25, 2002. Each of which is incorporated by reference herein.

### BRIEF DESCRIPTION OF THE DRAWINGS

FIGS. 1A-1C illustrate various states of the natural airways and the blood-gas interface.

FIG. 1D illustrates a schematic of a lung demonstrating a principle of the invention described herein.

FIG. 2A illustrates a side view of a conduit in an undeployed state.

FIG. 2B illustrates a side view of the conduit of FIG. 2A shown in a deployed shape.

FIG. 2C illustrates a front view of the conduit shown in FIG. 2B.

FIG. 2D is a cylindrical projection of the undeployed conduit shown in FIG. 2A.

FIG. 2E illustrates a side view of another conduit in an undeployed shape.

FIG. 2F illustrates a side view of the conduit of FIG. 2E in a deployed state.

FIG. 2G is a cylindrical projection of the undeployed conduit shown in FIG. 2E.

FIG. 3A illustrates a side view of another conduit having a tissue barrier in a deployed state.

FIG. 3B illustrates a side view of another conduit having a tissue barrier.

FIG. 3C is a front view of the conduit shown in FIG. 3B.

FIG. 3D illustrates a conduit positioned in a channel created in a tissue wall.

FIG. 3E is a cross sectional view of the conduit shown in FIG. 3B taken along line 3E-3E.

FIGS. 3F-3G depict another conduit including a membrane that supports a bioactive substance; the bioactive substance may be coated on the membrane.

FIGS. 4A-4C illustrate a method for deploying a conduit.

FIGS. 5A-5B illustrate a method for deploying a conduit at an angle.

FIGS. 6A-6E illustrate another technique for maintaining the patency of a channel through an airway wall.

FIG. 7 illustrates another implant having a distal region coated with a bioactive substance.

### DETAILED DESCRIPTION OF THE INVENTION

Described herein are devices (and methods) for improving the gas exchange in the lung. In particular, methods and devices are described that serve to maintain collateral openings or channels through an airway wall so that air is able to pass directly out of the lung tissue and into the airways. This facilitates exchange of oxygen into the blood and decompresses hyper inflated lungs.

By “channel” it is meant to include, but not be limited to, any opening, hole, slit, channel or passage created in the tissue wall (e.g., airway wall). The channel may be created in tissue having a discrete wall thickness and the channel may extend all the way through the wall. Also, a channel may extend through lung tissue which does not have well defined boundaries such as, for example, parenchymal tissue.

The channels may be maintained by preventing or inhibiting tissue from growing into or otherwise blocking the channel. Chemical, electrical, light, mechanical, or a combination of any two or more of these approaches may be performed to maintain the channel openings. For example, the channel walls may be treated with a bioactive agent which inhibits tissue growth. The bioactive agent may be delivered locally or systematically. Also, the channels may be treated with rf energy, heat, electrical energy, or radiation to inhibit tissue overgrowth. These treatments may be performed once, periodically, or in response to the severity of the channel blockage. For example, the tissue blockage may be periodically removed with a laser or another tissue-removal tool. Also, mechanical devices and instruments may be deployed in the channel to prevent tissue growth from blocking the channel. Mechanical devices include without limitation conduits, valves, sponges, etc. These mechanical devices may be deployed permanently or temporarily. If deployed temporarily, the devices are preferably left in the channel for a sufficient amount of time such that the channel tissue heals coaxially around the device.

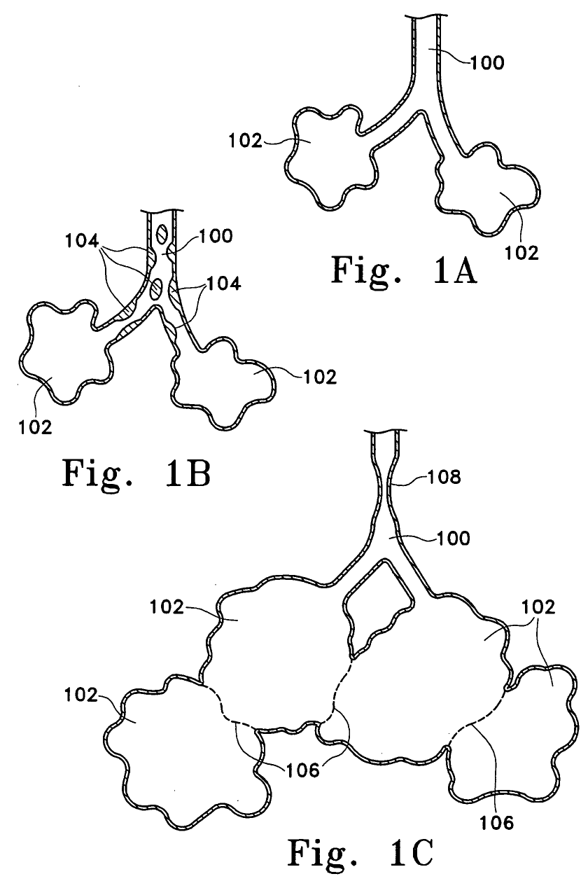

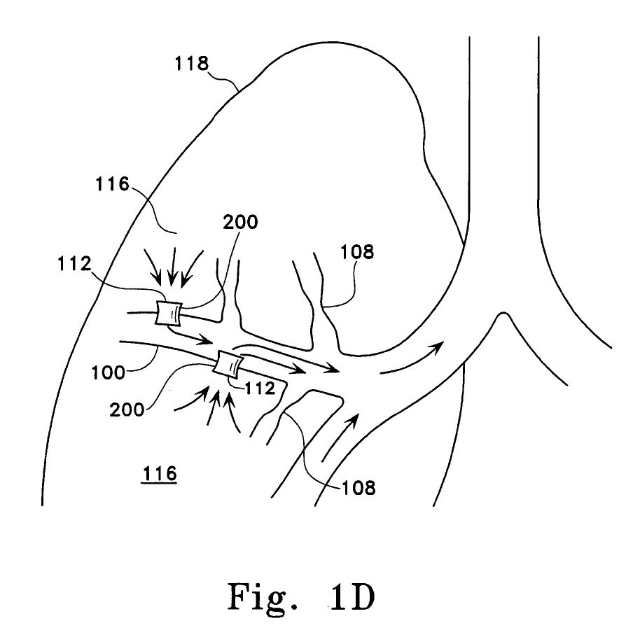

FIGS. 1A-1C are simplified illustrations of various states of a natural airway and a blood gas interface found at a distal end of those airways. FIG. 1A shows a natural airway 100 which eventually branches to a blood gas interface 102.

Although not shown, the airway comprises an internal layer of epithelial pseudostratified columnar or cuboidal cells. Mucous secreting goblet cells are also found in this layer and cilia may be present on the free surface of the epithelial lining of the upper respiratory airways. Supporting the epithelium is a loose fibrous, glandular, vascular lamina propria including mobile fibroblasts. Deep in this connective tissue layer is supportive cartilage for the bronchi and smooth muscle for the bronchi and bronchioles.

FIG. 1B illustrates an airway 100 and blood gas interface 102 in an individual having COPD. The obstructions 104 impair the passage of gas between the airways 100 and the interface 102. FIG. 1C illustrates a portion of an emphysematous lung where the blood gas interface 102 expands due to the loss of the interface walls 106 which have deteriorated due to a biochemical breakdown of the walls 106. Also depicted is a constriction 108 of the airway 100. It is generally understood that there is usually a combination of the phenomena depicted in FIGS. 1A-1C. Often, the states of the lung depicted in FIG. 1B and 1C may be found in the same lung.

FIG. 1D illustrates airflow in a lung 118 when conduits 200 are placed in collateral channels 112. As shown, collateral channels 112 (located in an airway wall) place lung tissue 116 in fluid communication with airways 100 allowing air to pass directly out of the airways 100 whereas constricted airways 108 may ordinarily prevent air from exiting the lung tissue 116. While the invention is not limited to the number of collateral channels which may be created, it is to be understood that 1 or 2 channels may be placed per lobe of the lung and perhaps, 2-12 channels per individual patient. However, as stated above, the invention includes the creation of any number of collateral channels in the lung. This number may vary on a case by case basis. For instance, in some cases in an emphysematous lung, it may be desirable to place 3 or more collateral channels in one or more lobes of the lung.

Although FIG. 1D depicts a mechanical approach to maintaining channels in the airway walls, the channel openings may be maintained using a variety of approaches or combinations of approaches.

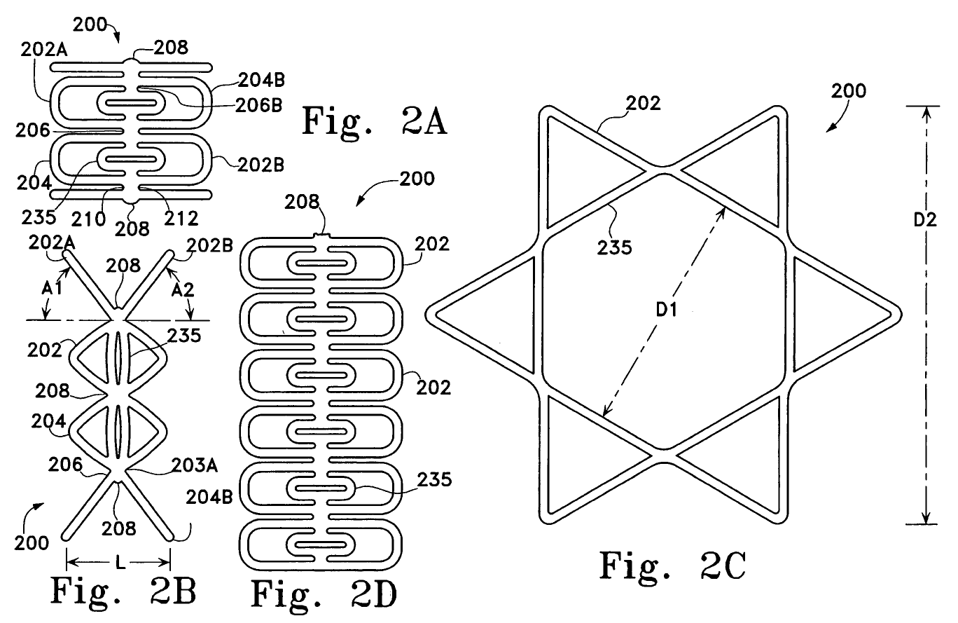

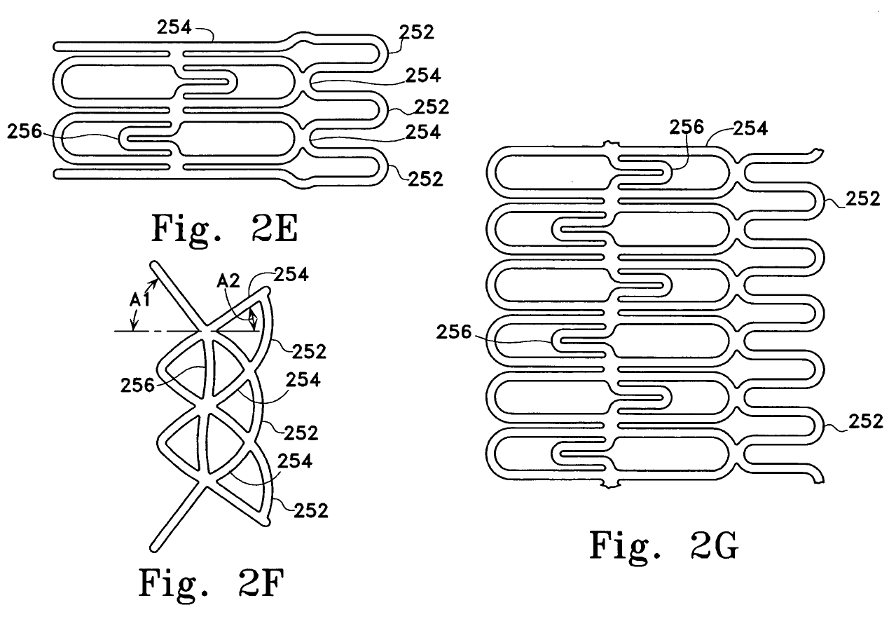

As shown in FIGS. 2A-2G, the conduits described herein generally include a center section 208 and at least one extension member (or finger) 202 extending from each end of the center section. The extension members, as will be discussed in more detail below, are capable of deflecting or outwardly bending to secure the conduit in an opening created in an airway wall thereby maintaining the patency of the opening. The extension members may deflect such that opposing extension members may form a V, U or other type of shape when viewed from the side.

Additionally, the conduits shown in FIGS. 2A-2G include a center-control segment 235, 256 which restricts or limits radial expansion of the center section. The center-control segments are adapted to straighten as the center section is radially expanded. Once the center-control segments become straight or nearly straight, radial expansion of the conduit is prevented. In this manner, the radial expansion of the conduit may be self controlled.

Conduit States

The conduits described herein may have various states (configurations or profiles) including but not limited to (1.) an undeployed state and (2.) a deployed state.

The undeployed state is the configuration of the conduit when it is not secured in an opening in an airway wall and, in particular, when its extension members (or fingers) are not outwardly deflected to engage the airway wall. FIG. 2A side view of a conduit 200 in an undeployed state. As shown in this figure, extension members 202A, 202B extend straight from the ends 210, 212 respectively of center section 208. The extension members shown in this example are parallel. However, the invention is not so limited and the extension members need not be parallel.

The deployed state is the configuration of the conduit when it is secured in a channel created in an airway wall and, in particular, when its extension members are outwardly bent to engage the airway wall such that the conduit is fixed in the opening. An example of a conduit in its deployed configuration is shown in FIGS. 2B and 2C. FIG. 2B is a side view of a conduit in its deployed state and FIG. 2C shows a front view of the conduit of FIG. 2B.

Center Section of the Conduit

As shown in FIGS. 2A-2D, the conduit includes a center section 208 having a short passageway. This center section may be a tubular-shaped open-frame (or mesh) structure having a plurality of ribs. Also, as explained in more detail below, the center section may be a sheet of material.

The axial length of the center section or passageway may be relatively short, In FIGS. 2A-2D, the passageway's length is about equal to the width of a wire segment or rib. Here, the center section serves as a bridge or junction for the extension members and it is not required to be long. The axial length of the passageway may therefore be less than 1 mm and even approach 0 mm. In one example, the length of the center section is less than twice the square root of a cross sectional area of the center section. However, the center section may also have passageways which have lengths greater than 1 mm.

The overall length (L) of the conduit may be distinguished from the length of the center section because the overall length includes the lengths of the extension members. Further, the overall length (L) is dependent on which state the conduit is in. The overall length of the conduit will typically be shorter when it is in a deployed state as shown in FIG. 2B than when it is in an undeployed state as shown in FIG. 2A. The overall length (L) for a deployed conduit may be less than 6 mm and perhaps, between 1 and 20 mm.

FIG. 2C shows a front view of the conduit 200 shown in FIG. 2B. FIG. 2C shows the passageway having a hexagonal (or circular) cross section. The cross-section, however, is not so limited. The cross section may be circular, oval, rectangular, elliptical, or any other multi-faceted or curved shape. The inner diameter (D₁) of the center section, when deployed, may range from 1 to 10 mm and perhaps, from 2 to 5 mm. Moreover, in some variations, the cross-sectional area of the passageway, when deployed, may be between 0.2 mm² to 300 mm² and perhaps between 3 mm² and 20 mm².

The diameter of the center section, when deployed, thus may be significantly larger than the passageway's axial length (e.g., a 3 mm diameter and an axial length of less than 1 mm). This ratio of the center section length to diameter (D1) may range from about 0:10 to 10:1, 0.1:6 to 2:1 andperhaps from 1:2 to 1:1. The diameter of the center section, when deployed, may also be nearly equal to the overall length (L) of the conduit 200. This overall length (L) to diameter (D1) ratio may range from 1:10 to 10:1, 1:6 to 2:1, and perhaps from 1:4 to 1:1. However, the invention is not limited to any particular dimensions or ratio unless so indicated in the appended claims. Rather, the conduit should have a center section such that it can maintain the patency of a collateral channel in an airway wall. The dimensions of the center section (and the conduit as a whole) may be chosen based on the tissue dimensions. When the channel is long in its axial length, for example, the length of the center section may likewise be long or identical to the channel's length.

Extension Members of the Conduit

As mentioned above, extending from the ends of the center section 208 are extension members 202A, 202B which, when the conduit is deployed, form angles A1, A2 with a central axis of the passageway. When viewed from the side such as in FIG. 2B, opposing extension members may have a V, U, or other shape. The extension members 202A, 202B may thus outwardly rotate until they sandwich tissue (not shown) between opposing extension members.

The angles A1, A2 may vary and may range from, for example, 30 to 150 degrees, 45 to 135 degrees and perhaps from 30 to 90 degrees. Opposing extension members may thus form angles A1 and A2 of less than 90 degrees when the conduit is deployed in a channel. For example, angles A1 and A2 may range from 30 to 60 degrees when the conduit is deployed.

The conduits of the present invention are effective and may maintain a surgically created opening despite not substantially sandwiching tissue between opposing extension members as described above. Additionally, it is not necessary for the conduits of the present invention to prevent air from flowing along the exterior of the conduit. That is, air may move into (and through) spaces between the exterior of the conduit and the interior wall of the tissue channel. Thus, fluidly sealing the edges of the conduit to prevent side flow or leakage around the conduit is not crucial for the conduits to be effective. However, the conduits of the present invention are not so limited and may reduce or eliminate side flow by, for example, increasing the angles A1 and A2 and adding sealant around the exterior of the conduit.

Moreover, the angle A1 may be different than angle A2. Accordingly, the conduit may include proximal extension members which are parallel (or not parallel) to the distal extension members. Additionally, the angle corresponding to each proximal extension member may be different or identical to that of another proximal extension member. Likewise, the angle corresponding to each distal extension member may be different or identical to that of another distal extension member.

The extension members may have a length between 1 and 20 mm and perhaps, between 2 and 6 mm. Also, with reference to FIG. 2C, the outer diameter (D₂) of a circle formed by the free ends of the extension members may range from 2 to 20 and perhaps, 3 to 10 mm. However, the invention is not limited to the dimensions disclosed above. Furthermore, the length of the distal extension members may be different than the length of the proximal extension members. The length of the distal extension members may be, for example, longer than that of the proximal extension members. Also, the lengths of each proximal extension member may be different or identical to that of the other proximal extension members. Likewise, the lengths of each distal extension member may be different or identical to that of the other distal extension members.

The number of extension members on each end of the center section may also vary. The number of extension members on each end may range from 2-10 and perhaps, 3-6. Also, the number of proximal extension members may differ from the number of distal extension members for a particular conduit. Moreover, the extension members may be symmetrical or non-symmetrical about the center section. The proximal and distal extension members may also be arranged in an in-line pattern or an alternating pattern. The extension members or the center section may also contain barbs or other similar configurations to increase adhesion between the conduit and the tissue. The extension members may also have openings to permit tissue ingrowth for improved retention.

The shape of the extension members may also vary. They may be open-framed and somewhat petal-shaped as shown in FIGS. 2A-2D. In these figures, the extension members 202A, 202B comprise wire segments or ribs that define openings or spaces between the members. However, the invention is not so limited and the extension members may have other shapes. The extension members may, for example, be solid or they may be filled.

In another variation the conduit is constructed to have a delivery state. The delivery state is the configuration of the conduit when it is being delivered through a working channel of a bronchoscope, endoscope, airway or other delivery tool. The maximum outer diameter of the conduit in its delivery state must therefore be such that it may fit within the delivery tool, instrument, or airway.

In one variation, the conduit is radially expandable such that it may be delivered in a smaller working channel of a scope while maximizing the diameter to which the conduit may expand upon deployment. For example, sizing a conduit for insertion into a bronchoscope having a 2 mm or larger working channel may be desirable. Upon deployment, the conduit may be expanded to have an increased internal diameter (e.g., 3 mm.) However, the invention is not limited to such dimensions. It is contemplated that the conduits 200 may have center sections that are expanded into a larger profile from a reduced profile, or, the center sections may be restrained in a reduced profile, and upon release of the restraint, return to an expanded profile.

Additionally, the conduit need not have a smaller delivery state. In variations where the center section is not able to assume a second smaller delivery profile, a maximum diameter of the first or deployed profile will be sufficiently small such that the conduit may be placed and advanced within an airway or a working channel of a bronchoscope or endoscope. Also, in cases where the conduit is self-expanding, the deployed shape may be identical to the shape of the conduit when the conduit is at rest or when it is completely unrestrained.

Control Members

The conduit 200 shown in FIGS. 2A-2D also includes diametriccontrol segments, tethers, or leashes 235 to control and limit the expansion of the center section 208 when deployed. This center-control segment 235 typically is shaped such that when the conduit radially expands, the center-control segment bends until it is substantially straight or no longer slack. Such a center-control segment 235 may be circular or annular shaped. However, its shape may vary widely and it may have, for example, an arcuate, semi-circular, V, or other type of shape which limits the expansion of the conduit.

Typically, one end of the center-control segment is attached or joined to the center section at one location (e.g., a first rib) and the other end of the center-control segment is connected to the center section at a second location (e.g., a rib adjacent or opposite to the first rib). However, the center-control segments may have other constructs. For example, the center-control segments may connect adjacent or non-adjacent center section members. Further, each center-control segment may connect one or more ribs together. The center-control segments may further be doubled up or reinforced with ancillary control segments to provide added control over the expansion of the center section. The ancillary control segments may be different or identical to the primary control segments.

FIG. 2B illustrates the conduit 200 in its deployed configuration. As discussed above, the center-control segments 235 may bend or otherwise deform until they maximize their length (i.e., become substantially straight) such as the center-control segments 235 shown in FIG. 2B. However, as discussed above, the invention is not so limited and other types of center-control segments may be employed.

As shown in FIGS. 2E-2G, control segments 252 may also be used to join and limit the expansion of the extension members 254 or the control segments may be placed elsewhere on the conduit to limit movement of certain features to a maximum dimension. By controlling the length of the control segments, the shape of the deployed conduit may be controlled. In the conduit shown in FIGS. 2E-2G, the conduit includes both center-control segments 256 and distal control segments 252. The center-control segments are arcuate shaped and join adjacent rib sections of the center section and the distal-control segments are arcuate and join adjacent distal extension members.

FIG. 2F illustrates the conduit in a deployed configuration and shows the various control members straightening as the extension members and center section deploy. The proximal extension members, however, are not restricted by a control member and consequently may be deflected to a greater degree than the distal extension members. Accordingly, a conduit having control members connecting, for example, regions of the center section and having additional control segments connecting extension members, may precisely limit the maximum profile of a conduit when it is deployed. This is desirable where overexpansion of the conduit is hazardous.

This also serves to control the deployed shape of the conduit by, for instance, forcing angle A1 to differ from angle A2. Using control segments in this manner can provide for cone-shaped conduits if the various types of control-segments have different lengths. For example, providing longer proximal-control segments than distal-control segments can make angle A1 larger than angle A2. Additionally, cylindrical-shaped conduits may be provided if the center-control segments and the extension-control segments are sized similarly such that angle A1 equals angle A2. Again, the control segments straighten as the conduit expands and the conduit is thus prevented from expanding past a predetermined amount.

The control segments, as with other components of the conduit, may be added or mounted to the center section or alternatively, they may be integral with the center section. That is, the control segments may be part of the conduit rather than separately joined to the conduit with adhesives or welding, for example. The control segments may also be mounted exteriorly or interiorly to the members to be linked. Additionally, sections of the conduit may be removed to allow areas of the conduit to deform more readily. These weakened areas provide another approach to control the final shape of the deployed conduit. Details for creating and utilizing weakened sections to control the final shape of the deployed conduit may be found in U.S. Pat. No. 09/947,144 filed on Sep. 4, 2001.

Manufacture and Materials

The conduit described herein may be manufactured by a variety of manufacturing processes including but not limited to laser cutting, chemical etching, punching, stamping, etc. For example, the conduit may be formed from a tube that is slit to form extension members and a center section between the members. One variation of the conduit may be constructed from a metal tube, such as stainless steel, 316L stainless steel, titanium, titanium alloy, nitinol, MP35N (a nickel-cobalt-chromium-molybdenum alloy), etc. Also, the conduit may be formed from a rigid or elastomeric material that is formable into the configurations described herein. Also, the conduit may be formed from a cylinder with the passageway being formed through the conduit. The conduit may also be formed from a sheet of material in which a specific pattern is cut. The cut sheet may then be rolled and formed into a tube. The materials used for the conduit can be those described above as well as a polymeric material, a biostable or implantable material, a material with rigid properties, a material with elastomeric properties, or a combination thereof. If the conduit is a polymeric elastic tube (e.g. a thermoplastic elastomer), the conduit may be extruded and cut to size, injection molded, or otherwise formed.

Additionally, the conduits described herein may be comprised of a shape memory alloy, a super-elastic alloy (e.g., a NiTi alloy), a shape memory polymer, or a shape memory composite material. The conduit may be constructed to have a natural self-assuming deployed configuration, but is restrained in a pre-deployed configuration. As such, removal of the restraints (e.g., a sheath) causes the conduit to assume the deployed configuration. A conduit of this type could be, but is not limited to being, comprised from an elastic polymeric material, or shape memory material such as a shape memory alloy. It is also contemplated that the conduit could comprise a shape memory alloy such that, upon reaching a particular temperature (e.g., 98.5° F.), it assumes a deployed configuration.

Also, the conduit described herein may be formed of a plastically deformable material such that the conduit is expanded and plastically deforms into a deployed configuration. The conduit may be expanded into its expanded state by a variety of devices such as, for example, a balloon catheter.

The conduit's surface may be modified to affect tissue growth or adhesion. For example, an implant may comprise a smooth surface finish in the range of 0.1 micrometer to 0.01 micrometer. Such a finish may serve to prevent the conduit from being ejected or occluded by tissue overgrowth. On the other hand, the surface may be roughened or porous. The conduit may also comprise various coatings and tissue barriers as discussed below.

Tissue Barrier

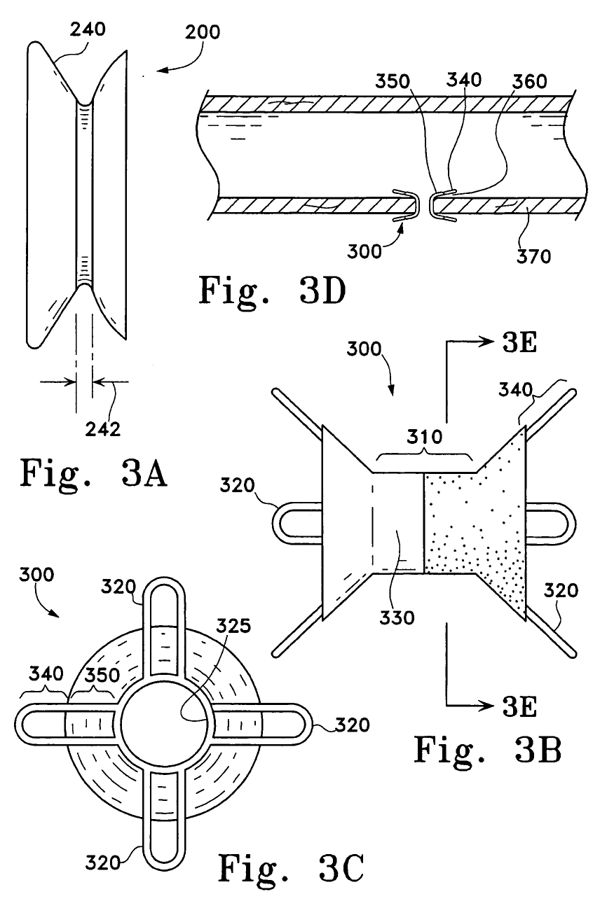

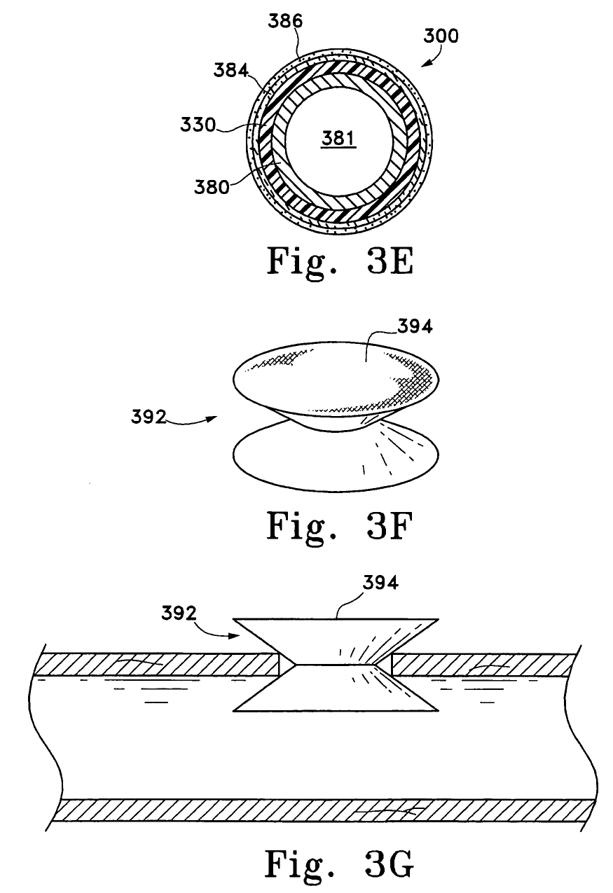

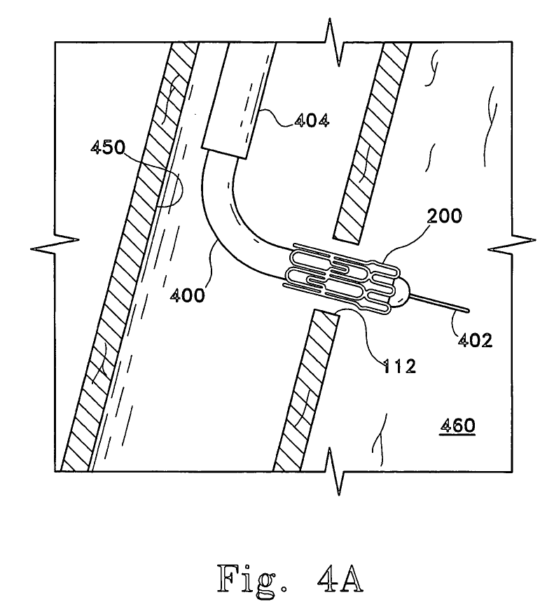

FIG. 3A illustrates another variation of a conduit 200 having a tissue barrier 240. The tissue barrier 240 prevents tissue ingrowth from occluding the collateral channel or passage of the conduit 200. The tissue barrier 240 may coaxially cover the center section from one end to the other or it may only cover one or more regions of the conduit 200. The tissue barrier may completely or partially cover the conduit so long as the ends are at least partially open. The tissue barrier 240 may be located about an exterior of the conduit's surface, about an interior of the conduit's surface, or the tissue barrier 240 may be located within openings in the wall of the conduit's surface. Furthermore, in some variations of the invention, the center section 208 itself may provide an effective barrier to tissue ingrowth. The tissue barrier, of course, should not cover or block the entrance and exit of the passageway such that air is prevented from passing through the conduit's passageway. However, in some constructs, the tissue barrier may partially block the entrance or exit of the passageway so long as air may continue to pass through the conduit's passageway.

The tissue barrier may be formed from a material, mesh, sleeve, or coating that is a polymer or an elastomer such as, for example, silicone, fluorosilicone, polyurethane, PET, PTFE, or expanded PTFE. Other biocompatible materials will work, such as a thin foil of metal, etc. The coatings may be applied, for example, by either dip coating, molding, spin-coating, transfer molding or liquid injection molding. Alternatively, the tissue barrier may be a tube of a material and the tube is placed either over and/or within the conduit. The tissue barrier may then be bonded, crimped, heated, melted, shrink fitted or fused to the conduit. The tissue barrier may also be tied to the conduit with a filament of, for example, a suture material.

Still other techniques for attaching the tissue barrier include: solvent swelling applications and extrusion processes; wrapping a sheet of material about the conduit, or placing a tube of the material about the conduit and securing the tube to the conduit. The tissue barrier may be secured on the interior of the conduit by positioning a sheet or tube of material on the inside of the center section and securing the material therein.

The tissue barrier may also be formed of a fine mesh with a porosity or treatment such that tissue may not penetrate the pores. For example, a ChronoFleX™ DACRON® or TEFLON® mesh having a pore size of 100-300 microns may be saturated with collagen or another biocompatible substance. This construct may form a suitable tissue barrier. The mesh may be coaxially attached to a frame such as the open frame structures disclosed above. Still other suitable frames include a continuous spiral metallic or polymeric element. Given the mesh's radial strength or lack thereof, the use of a reinforcement element serves to prevent the implant from collapsing. Also, as described below, other substances may be applied to the exterior surface of the conduit to control elution of various medicines.

FIGS. 3B and 3C respectively illustrate a side view and a front view of another conduit 300 having a partial tissue barrier coating. The conduit 300 includes a center section 310, a plurality of extension members 320, and a partial tissue barrier 330. The conduit 300 is thus different than that shown in FIG. 3A in that the center section is longer and that the tissue barrier 330 only partially covers the extension members 320. In particular, the center section 310 shown in FIGS. 3B-3C is cylindrical or tubular-shaped. This shape may be advantageous when a relatively long passageway is desired. Also, it is to be understood that the overall (or three dimensional) shape of the center section, when deployed, is not limited to the shape shown here. Rather, it may have various shapes such as, for example, rectangular, tubular, conical, hour-glass, hemi-toroidal, etc.

Additionally, the tissue barrier 330 covers only a first region 350 of the extension members and leaves a second region 340 of the extension members uncovered. The second or free region 340 of the extension members 320 is shown as being open-framed. However, the invention is not so limited. The second region of the extension members may be solid and it may include indentations, grooves, and recesses for tissue ingrowth. Also, the extension members may include small holes for tissue ingrowth. For example, the second region of the extension members may have a dense array of small holes. In any event, the conduits described herein may include at least one region or surface which is susceptible to tissue ingrowth or is otherwise adherent to the tissue. Accordingly, tissue ingrowth at the second region 340 of the extension members is facilitated while tissue growth into the passageway 325 is thwarted.

As shown in FIG. 3D, tissue growth 360 into the uncovered region 340 further secures the extension members to the tissue wall 370. Free region 340 of the extension members may also include tissue growth substances such as epithelial growth factors or agents to encourage tissue ingrowth. Accordingly, conduit 300 may be configured to engage the tissue wall 370 as well as to allow tissue to grow into predetermined regions of the conduit.

Visualization Feature

The conduit shown in FIG. 3A also includes a visualization ring or marker 242. The marker 242 is visually apparent during a procedure. The marker is observed as the conduit is placed in a collateral channel and, when the marker is even with the opening of the channel, the conduit may be deployed. In this manner, the visualization feature facilitates alignment and deployment of the conduits into collateral channels.

The visualization ring or mark may be a biocompatible polymer and have a color such as white. Also, the visualization feature may protrude from the center section or it may be an indentation(s). The visualization mark may also be a ring, groove or any other physical feature on the conduit. Moreover, the visualization feature may be continuous or comprise discrete segments (e.g., dots or line segments).

The visualization feature may be made using a number of techniques. In one example, the mark is a ring formed of silicone and is white. The polymeric ring may be spun onto the tissue barrier. For example, a clear silicone barrier may be coated onto the conduit such that it coaxially covers the extension members and the center section as shown in FIG. 3A. Next, a thin ring of white material such as a metal oxide suspended in clear silicone may be spun onto the silicone coating. Finally, another coating of clear silicone may be applied to coat the white layer. The conduit thus may include upwards of 1-3 layers including a tissue barrier, a visualization mark layer, and a clear outer covering.

The shape of the visualization mark is not limited to a thin ring. The visualization mark may be large, for example, and cover an entire half of the conduit as shown in FIG. 3B. The visualization mark may, for example, be a white coating disposed on the proximal or distal half of the conduit. The visualization mark thus may extend from an end of the extension members to the center section of the conduit. As explained in more detail below, when such a device is deposited into a channel created in lung tissue, the physician may observe when one-half of the conduit extends into the channel. This allows the physician to properly actuate or deploy the conduit to secure the conduit in the tissue wall.

Accordingly, the visualization member is made visually apparent for use with, for example, an endoscope. The visualization feature, however, may also be made of other vision-enhancing materials such as radio-opaque metals used in xray detection. It is also contemplated that other elements of the conduit can include visualization features such as but not limited to the extension members, tissue barrier, control segments, etc.

Bioactive Agents

Medicines and bioactive agents may be delivered locally or systematically to inhibit tissue growth or otherwise prevent tissue from blocking a surgically created channel. In the event a bioactive agent is delivered systematically, the agent may be inhaled, ingested, absorbed, injected, etc.

In the event the bioactive agent or medicine is delivered locally, the agent may be delivered using a catheter or other delivery tool which can access the airways of the lung. Also, as described in more detail below, the medicine may be delivered in combination with an implantable device such as a conduit, plug, or another device which is deployed temporarily or permanently in the channel. Thus, the medicine may be associated with an implant or it may be delivered separately through, for example, a delivery instrument such as a delivery catheter.

The bioactive substances are intended to interact with the tissue of the surgically created channels and in particular, lung tissue. These substances may interact with the tissue in a number of ways. They may, for example, 1.) accelerate cell proliferation or wound healing to epithelialize or scar the walls of the surgicallycreated channel to maintain its patent shape or 2.) the substances may inhibit or halt tissue growth when a channel is surgically created through an airway wall such that occlusion of the channel due to tissue overgrowth is prevented. Additionally, other bioactive agents may inhibit wound healing such that the injury site (e.g., the channel or opening) does not heal leaving the injury site open and/or inhibit infection (e.g., reduce bacteria) such that excessive wound healing does not occur which may lead to excessive tissue growth at the channel thereby blocking the passageway. Not wishing to be limited to theory, there may be other explanations why certain bioactive substances have various therapeutic uses in the lung tissue. Again, the bioactive substances are intended to maintain the patency of surgically created channels or otherwise prevent the channels from being blocked.

A variety of bioactive substances may be used alone or in combination with the devices described herein. Examples of bioactive substances include, but are not limited to, antimetabolites, antithrobotics, anticoagulants, antiplatelet agents, thorombolytics, antiproliferatives, antinflammatories, agents that inhibit hyperplasia and in particular restenosis, smooth muscle cell inhibitors, growth factors, growth factor inhibitors, cell adhesion inhibitors, cell adhesion promoters and drugs that may enhance the formation of healthy neointimal tissue, including endothelial cell regeneration. The positive action may come from inhibiting particular cells (e.g., smooth muscle cells) or tissue formation (e.g., fibromuscular tissue) while encouraging different cell migration (e.g., endothelium, epithelium) and tissue formation (neointimal tissue).

Still other bioactive agents include but are not limited to analgesics, anticonvulsives, anti-infectives (e.g., antibiotics, antimicrobials), antineoplastics, H2 antagonists (Histamine 2 antagonists), steroids, non-steroidal anti-inflammatories, hormones, immunomodulators, mast cell stabilizers, nucleoside analogues, respiratory agents, antihypertensives, antihistamines, ACE inhibitors, cell growth factors, nerve growth factors, anti-angiogenic agents or angiogenesis inhibitors (e.g., endostatins or angiostatins), tissue irritants (e.g., a compound comprising talc), poisons (e.g., arsenic), cytotoxic agents (e.g., a compound that can cause cell death), various metals (silver, aluminum, zinc, platinum, arsenic, etc.), or a combination of any of the agents disclosed herein.

Additionally, lung-active substances may be used to prevent closure including lung-active substances which affect the rate of wound healing in lung tissue. Examples of agents include but are not limited to: agents that affect the growth or production (e.g., facilitate or retard) of epithelial cuboidal cells, epithelial pseudostratified columnar cells, and other tissues derived from the mesoderm embryonic germ layer.

Examples of agents include pyrolitic carbon, titanium-nitride-oxide, taxanes, fibrinogen, collagen, thrombin, phosphorylcholine, heparin, rapamycin, radioactive 188Re and 32P, silver nitrate, dactinomycin, sirolimus, everolimus, Abt-578, tacrolimus, camptothecin, etoposide, vincristine, mitomycin, fluorouracil, or cell adhesion peptides. Taxanes include, for example, paclitaxel, 10-deacetyltaxol, 7-epi-10-deacetyltaxol, 7-xylosyl-10-deacetyltaxol, 7-epi-taxol, cephalomannine, baccatin III, baccatin V, 10-deacetylbaccatin III, 7-epi-10-deacetylbaccatin III,docetaxel.

Of course, bioactive materials having other functions can also be successfully delivered in accordance with the present invention. For example, an antiproliferative agent such as methotrexate will inhibit over-proliferation of smooth muscle cells and thus inhibit restenosis. The antiproliferative is desirably supplied for this purpose until the tissue has properly healed. Additionally, localized delivery of an antiproliferative agent is also useful for the treatment of a variety of malignant conditions characterized by highly vascular growth. In such cases, an implant such as a conduit could be placed in the surgically created channel to provide a means of delivering a relatively high dose of the antiproliferative agent directly to the target area. A vasodilator such as a calcium channel blocker or a nitrate may also be delivered to the target site. The agent may further be a curative, a pre-operative debulker reducing the size of the growth, or a palliative which eases the symptoms of the disease. For example, tamoxifen citrate, Taxol® or derivatives thereof Proscar®, Hytrin®, or Eulexin® may be applied to the target site as described herein.

In the event that poisonous and toxic compounds are delivered, they should be controlled so that inadvertent death of tissue does not occur. The poisonous agent should be delivered locally or only be effective locally. One method for delivering the bioactive agent locally is to associate the bioactive agent with an implant. For example, the conduits described herein may include a bioactive substance or medicine deposited onto the interior, the exterior, or both the interior and exterior surfaces of the conduit. The bioactive substance may remain on the conduit so that it does not leach. Cells that grow into the surgically created channel contact the poison and die. Alternatively, the bioactive agent may be configured to gradually elute as discussed below.

A cross section of a conduit 300 having a bioactive modified surface is shown in FIG. 3E. In particular, the conduit 300 comprises an inner frame layer or ribs 380 which define a passageway 381 for air to flow through. Coaxially surrounding the frame 380 is a tissue barrier 330. Additionally a visualization coating 384 is disposed on the tissue barrier 330. The visualization coating 384 is deposited as described above. A bioactive substance 386 is deposited on the visualization layer either directly or via a binding layer as described below. In this manner, the bioactive substance is disposed on an exterior surface of the conduit and contacts tissue or elutes into the tissue when the device is deployed in a channel. However, it is contemplated that additional layers may be added such as, for example, an additional silicone or bioabsorbable layer over (or in combination with) the visualization layer.

Also the order of the layers may be different than that described above. For example, the visualization layer may be disposed over the bioactive layer. Also, not all coatings and materials shown in FIG. 3E are necessary to carry out the present invention. For instance, the bioactive substances in some cases may be deposited directly on the open-frame 380.

The bioactive layer may also serve as the visualization coating or tissue barrier in some instances. For example, silicone and one or more bioactive substances may be mixed together and disposed on the conduit as a single coating. The single integral layer (or matrix) may serve both to physically and chemically prevent tissue from filling the conduit's passageway. It may also be visually apparent during a procedure.

The bioactive compounds may be combined, impregnated, absorbed or attached to any of the implants described herein. For example, a bioactive substance may be impregnated into a polymeric conduit or a metal conduit having a polymeric coating. These polymer systems hold the drug, allowing it to gradually elute or slowly leach from the polymer body or coating. Useful polymer systems include a polymer that is biocompatible and minimizes irritation to the tissue wall when the conduit is implanted. The polymer may be either a biostable or a bioabsorbable polymer depending on the desired rate of release or the desired degree of polymer stability, but a bioabsorbable polymer, unlike a biostable polymer, will not be present long after implantation to cause any adverse, chronic local response.

Examples of bioabsorbable polymers include but are not limited to poly(L-lactic acid), polycaprolactone, poly(lactide-co-glycolide), poly(hydroxybutyrate), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g., PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, fluorosilicones, and polyesters could be used. Also, hydrogels may be used to carry the drug.

Examples of other types of polymers that may be useful include but are not limited to polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrilestyrene copolymers, ABS resins, and ethylene-vinyl acetate copolymers; polyamides, such as Nylon 66 and polycaprolactam; alkyd resins, polycarbonates; polyoxymethylenes; polyimides; polyethers; epoxy resins, polyurethanes; rayon; rayon triacetate; cellulose, cellulose acetate, cellulose butyrate; cellulose acetate butyrate; cellophane; cellulose nitrate; cellulose propionate; cellulose ethers; and carboxymethyl cellulose. It may be possible to dissolve and cure (or polymerize) these polymers on the conduit so that they do not leach into the tissue and cause any adverse effects on the tissue.

The conduits may be coated or impregnated variously. For example, a polymer solution may be applied to the conduit and the solvent allowed to evaporate, thereby leaving on the conduit surface a coating of the polymer and the therapeutic substance. Typically, the solution can be applied to the conduit by either spraying the solution onto the conduit or immersing the conduit in the solution. In either a coating applied by spraying or by immersion, multiple application steps are generally desirable to provide improved coating uniformity and improved control over the amount of therapeutic substance to be applied to the conduit.

The bioactive substances may be deposited on the exterior surface of the conduit evenly or in discrete (intermittent) amounts. The thickness of the coatings may be uniform or the thickness may vary across certain regions of the conduit. This may provide higher therapeutic doses corresponding to certain regions of the injury site. For example, it may be desirable to provide a higher concentration of a bioactive substance near the ends of the conduit rather than in the center section.

The bioactive coatings may be selectively applied by spraying the bioactive substance onto uncovered regions of the conduit. For example, the bioactive substances may be disposed on at least a portion of the tissue barrier, the open-frame, or mesh structure. The substances may also be applied by dipping, painting, printing, and any other method for impregnating or depositing a substance onto the conduit surface.

FIGS. 3F-3G depict another conduit 392 that supports a bioactive substance. In particular, conduit 392 includes a membrane, mesh, or framework 394 that covers one or both ends of the conduit. A bioactive substance is associated with the mesh. For example, a bioactive substance may coat the mesh. FIG. 3G shows the conduit deployed in an airway and the mesh 394 is facing the parenchyma. The coated mesh may be present on one end or both ends of the conduit. Also, a wide variety of bioactive substances may be applied to the mesh and the conduit. Antiproliferative or cytotoxic substances, for example, may be applied to any portion of the mesh, the inside of the conduit, the outside of the conduit, or both the inside and outside of the conduit.

Binding or tie layer materials may be applied to the exterior surface of the conduit upon which the bioactive agents are deposited. Cross-linked polymers and or biodegradable polymers such as, for example, chondroitin sulfate, collagen and gelatin may be applied to the exterior surface of the conduit prior to depositing other substances.

Solvent-type coatings, gels or mediums may also be used in combination with a conduit body. The advantage of a solvent system stems from the unique characteristics of the airway. Amongst other things, the airway is specialized to transport air and mucus. In an airway, as opposed to a blood vessel, diffusion of a drug to a target site may be slow since there is not a natural flowing liquid (such as blood in a blood vessel) to dissolve the polymer and disperse the drug into the tissue. For this reason, a paste, viscous solvent, or gel may desirably be disposed on the exterior of the conduit to facilitate the diffusion of drugs into nearby tissue.

Thin coatings may also be deposited on the conduit surfaces. Thin coatings of, for example, tantalum may be applied to the conduits to increase the radiopacity, to change the charge on the surface of the conduit, to provide a tie layer upon which other agents may be deposited, or for other reasons. Physical vapor deposition such as plasma deposition, vapor phase deposition, ion plating and ion implantation may be useful to apply such a layer to the conduit frame or mesh structure.

Additionally, the exterior surface of the conduit may be treated via etching processes or with electrical charge to encourage binding of the bioactive substances to the conduit. The exterior surface may also be roughened to enhance binding of the medicine to the surface as discussed in U.S. patent application Ser. No. 2002/0098278. See also U.S. patent application Ser. Nos. 2002/0071902, 2002/0127327 and U.S. Pat. No. 5,824,048 which discuss various techniques for coating medical implants.

Although the conduit may comprise a frame or body with a bioactive matrix disposed or otherwise associated therewith, the invention is not so limited. In one variation, a polymer and medicine are combined in a mixture (e.g., a homogeneous mixture) and formed into a conduit. The medicine is thus present throughout the conduit body and can slowly leach into surrounding tissue when implanted.

In another variation, a conduit includes hollowed portions that contain medicine. For example, the conduit struts, ribs, or legs may be hollow (or otherwise have internal cavities). Microholes are provided and extend to the hollowed portion or cavities. Medicine contained within these hollowed portions may elute over time through the holes to the target tissue areas. Moreover, a capsule having microholes may be delivered into a channel. The capsule may be filled with a medicine compound such that medicine is delivered through the microholes into the target tissue. The capsule itself may be formed of a bioabsorbable material or it may be formed of another material. If necessary, the capsule may be physically removed when the tissue has healed in the proper shape. The microholes may be drilled or otherwise formed. Also, the skin of the capsule or body of the conduit containing the medicine may be a mesh or porous structure such that the medicine may leach into the target tissue.

In another variation, the conduit is formed of bi-metals that galvanically corrode to release a medicine contained therein. The medicine may be, for example, contained in a cavity that is covered by a corrodable metal. The metals are dissimilar and in contact with one another such that in the presence of moisture or an electrolyte, electrons flow from one metal to the other. This leads to corrosion (galvanic corrosion) of the anode metal which controls release of the medicine. As more corrosion occurs, more medicine is released from the conduit. The metals may be positioned in layers, side-by-side, or otherwise assembled together such that the above described corrosion phenomena may occur.

In yet another variation, the conduit includes a battery eluting scheme. In particular, a battery may be built up on the conduit to provide a current and to control the medicine dosage. The medicine mixture may be coated on a cathode surface of the conduit and the cathode may be formed of, for example, a carbon substance. A perforated zinc or other suitable metal layer is then disposed over the medicine coating. The medicine coating is selected such that it may transfer ions from the anode to the cathode. As ions are transferred, the anode corrodes increasing the rate at which the medicine is released. The metals used for the battery are preferably biocompatible or bioabsorbable metals.

Additionally, the cathode or inner metal layer may be the conduit frame itself. The medicine medium or paste may be disposed over the conduit body. An outer perforated, less noble, metal may be disposed over the medicine paste. When deployed in a surgically-created channel having moisture a circuit is created. Ions from the anode move to the cathode corresponding to the number of electrons carried externally through the circuit. As the anode metal corrodes, the conduit will deliver more medicine. In this manner, an electrically enhanced drug-delivery conduit may maintain the patency of a channel.

Still other coatings for controlled release of medicines, negatively charged layers and hydrophobic layers, drug saturated biodegradable matrixes, drug eluting electrodeposited compounds, diffusion barriers, and other types of medicine delivery schemes may be used in connection with the present invention as described in U.S. patent application Ser. Nos.: 2002/0123801, 2002/0119178, 2002/0032477, 2002/0099434, 2002/0061326, 2002/0091433, 2002/0032414, 2002/0032434, 2001/0014717.

While conduits with drug eluting properties may also yield some effectiveness in maintaining the patency of a channel, in another example of the invention, it was found that delivery of a medicine or bio-active agent (hereafter collectively referred to as “substance”) could be delivered to or near the site of the channel using an eluting delivery system that permits a controlled release of the substance to be dispersed in the local area adjacent to the channel but not necessarily concentrated in the area immediate to the conduit. For instance, cardiovascular drug eluting stent applications rely upon the drug to be delivered through a delivery agent, usually a polymeric carrier, that is attached to the stent. Accordingly, the drug delivery is concentrated in the area of the stent. Given the nature of the vasculature, where the stent is subject to continuous blood flow, it may be desirable to provide a concentrated delivery of the drug to only the area of the stent as the bloodstream would disperse the drug if it were deployed in areas beyond the stent.

In contrast, when applying a substance to the lungs/airways to maintain the opening of the channel, it may also be desirable to locally deliver the substance beyond the immediate range of the conduit or stent. Because the conduit is placed in the airway, through the airway wall, and into lung parenchyma, it may be desirable to prevent tissue growth in areas beyond where the conduit actually contacts tissue. It was found during studies that the delivery of a substance, having a polymeric carrier, to an area in and around the channel provided extended results in maintaining the patency of the conduit within the channel.

The use of a polymeric carrier that is separate from the device yields an additional advantage in the selection of the polymeric carrier. For example, a polymeric carrier may be used although it is not suitable to forming the bulk structure required to coat and adhere to the conduit. In such a case, it is possible to select a polymeric carrier with more desirable drug carrier properties than another polymeric carrier that must also be attached to the conduit. For example, the polymeric carrier may be able to hold more of the substance thereby allowing for a uniform continuous effective dose to be delivered in the treatment area. Furthermore, the polymeric carrier may be selected to have properties that allow for the carrier to adhere to the lung tissue so that movement of the lung tissue, mucous, or airflow, do not readily disperse the carrier/substance from the area of desired treatment.

In one variation of the invention, a precipitated form of polymer coated paclitaxel may be particularly effective. For example, a 6 mg/ml of Cremophor EL (BASF Corporation) solution was diluted to 2 mg/ml and a sufficient period of time elapsed to form the precipitate. It is believed that upon examination of the precipitate, that in the diluted solution the polymer in the Cremophor EL encapsulated the paclitaxel molecules as the two precipitated out of the solution. The polymer from the Cremophor may also be conducive to adhering to lung tissue causing the precipitate to adhere to the tissue to release the drug in a controlled manner over the zone in which the solution was applied. It is believed that any class of polyethylene oxide, polypropylene oxide, polymers used as emulsifiers, polyvinyl acetate, and polymers that have a hydrophilic head and hydrophobic tail, would serve as acceptable delivery agents.

It should be noted that a conduit may also be loaded with a precipitate (e.g., polymer coated substance such as that discussed above) where upon delivery of the conduit the precipitate may migrate from the conduit to the areas adjacent to the channel. Moreover, it is contemplated that delivery of any of the substances discussed herein may be combined with a polymeric delivery agent where the combination is then delivered to the area surrounding the channel. However, it may be desirable to use substances having low solubility.

To reiterate, the present invention may include bioactive polymer systems associated with an implant or conduit to provide a controlled release of a medicine. The implant itself may be formed of a polymer matrix, or the conduit may comprise a polymer matrix coating which is deposited on a frame or mesh. In any case, the conduit provides for delivery of the bioactive agent to the surgically created channel.

Implants within Conduits

The conduits may further comprise various structures deposited within the passageway. For example, a conduit may include a one-way valve. The valve may be positioned such that it permits expiration of gas from lung tissue but prevents gas from entering the tissue. The valve may be placed anywhere within the passageway of the conduit. The valve may also be used as bacterial in-flow protection for the lungs. The valve may also be used in combination with a bioactive or biostable tissue barrier/matrix and the tissue barrier may be disposed coaxially about the conduit. Various types of one way valves may be used as is known to those of skill in the art.

In another variation, a second conduit is deployed within the first conduit. See, e.g., U.S. patent application Ser. No. 2001/0044650 which discloses a second stent implanted within a first stent which has been previously implanted in a body vessel.

In another variation, biodegradable or removable sponges are associated with the conduit. In particular, the sponge may be positioned within the passageway of the conduit (or may constitute the whole conduit) to prevent tissue overgrowth or to prevent tissue from otherwise blocking the passageway of the conduit. The sponge may be removed after a period of time sufficient to allow the tissue to heal. The sponge may be a natural or synthetic sponge. Examples of sponges include porous plastics, open cell foam, rubber, cellulose and other porous absorbent materials. The sponges typically have pores and the pore size may be that found in conventional sponges including pore sizes as low as 200 microns. See, for example, RAMERFOAM®. The pore size may be uniform or non-uniform throughout the sponge. If a biodegradable sponge is deployed in the channel or the passageway of the conduit, the device does not need to be removed as it will degrade with time. The sponge should be present, in any case, long enough to set the tissue or block the tissue overgrowth. The sponges may also be drug loaded with substances that prevent or inhibit tissue overgrowth. Bioactive substances as described above may be loaded onto the sponges by dipping the sponges in a solution and allowing the sponges to dry. Bioactive substances may be loaded onto the sponges in other manners as is known to those of ordinary skill in the art.

In another variation, the conduits are associated with graftable tissue. The tissue may be excised from human and non-human mammals (i.e., it may be allogeneic or xenogeneic). For example, a vein graft may be attached to the exterior surface, interior surface, or both surfaces of the conduit. The graftable tissue may be from a vein or airway. It may be tissue from the same individual (autologous/autograft) or another individual. It may be allogeneic or autologous. The graftable tissue may be coaxially bonded to a mesh or frame structure having a plurality of ribs such that graftable tissue forms an exterior surface of the conduit or the graftable tissue may be coaxially bonded to a hollow body structure having a wall such that the graftable tissue forms an exterior surface of the conduit. The graft may be inverted, or turned inside out, to facilitate placing the epithelial or endothelial surface of the graft on the outside of the conduit assembly.

Conduit Deployment Method

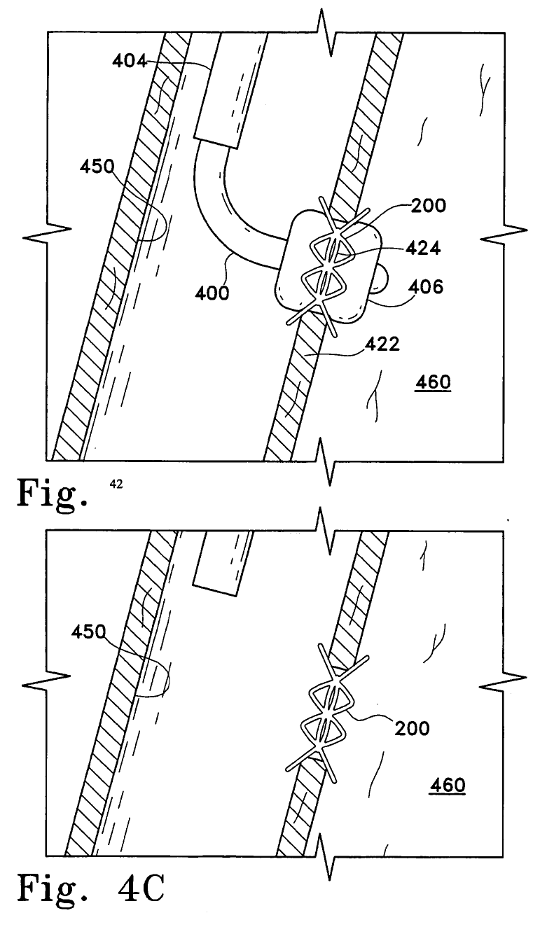

FIGS. 4A-4C illustrate a way to deploy a conduit in a channel. Referring to FIG. 4A, a delivery device 400 is loaded with a conduit 200. An access device 404 (e.g., an endoscope, a bronchoscope, or other device) may optionally be used to place the delivery device 400 into a collateral channel 112. A guide wire 402 may be used to place the delivery device 400 into the collateral channel 112. The guide wire 402 may be a conventional guide-wire or it may simply be comprised of a super-elastic material. The use of a guide wire is optional as the invention contemplates placement of the conduit 200 using only the delivery device 400.

FIG. 4A also illustrates articulation (or bending) of the deliver device 400 to access the collateral channel 112. However, the invention also contemplates articulation of the access device 404. The access device 404 may be articulated such that the delivery device 400 may advance straight into the collateral channel 112. Accordingly, the delivery device 400 may exit straight from the access device 404 or it may be articulated into the opening.

FIG. 4B illustrates deployment of the conduit 200. In particular, balloon member 406 is shown in an expanded state resulting in (1.) the conduit's center section being radially expanded and (2.) the conduit's extension members being outwardly deflected such that opposing extension members sandwich portions of the tissue wall 422. Diametric-control members 424 are also shown in this figure. The diametric or center-control segments limit the center section's radial expansion. In this manner, conduit 200 is securely placed in the channel to maintain a passageway through the airway wall 422.

FIG. 4C illustrates the deployed conduit 200 once the delivery device 400 is removed from the site.

It should be noted that deployment of conduits is not limited to that shown in FIGS. 4A-4C, instead, other means may be used to deploy the conduit. For example, spring-loaded or shape memory features may be actuated by mechanical or thermal release and unlocking methods. Additionally, mechanical wedges, lever-type devices, scissors-jack devices, open chest surgical placement and other techniques may be used to deploy the conduit. Again, the conduit 200 may be comprised of an elastic or super-elastic material which is restrained in a reduced profile for deployment and expands to its deployed state upon mechanical actuator or release.

In use, the conduit 200 is deployed with the distal side towards the parenchymal tissue 460 while the proximal side remains adjacent or in the airway 450. Of course, where the proximal and distal extension members are identical, the conduit may be deployed with either side towards the parenchymal tissue.

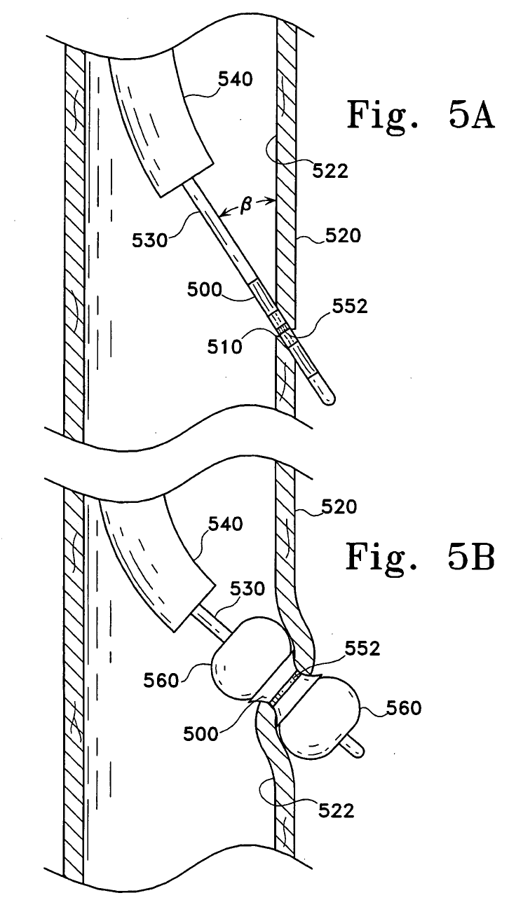

FIGS. 5A-5B illustrate another example of deploying a conduit 500 in a channel 510 (or opening) created in a tissue wall 520. Referring to FIG. 5A, a delivery tool 530 carrying a deployable conduit 500 is inserted into the channel 510. The delivery tool 530 is extended straight from an access catheter 540 such that the delivery tool forms an angle [symbol] with the tissue wall 520. It is to be understood that while the tissue wall of airway 522 is shown as being thin and well defined, the present invention may be utilized to maintain the patency of channels and openings which have less well defined boundaries. The delivery tool is further manipulated until the conduit is properly positioned which is determined by, for example, observing the position of a visualization mark 552 on the conduit relative to the opening of the channel 510.

FIG. 5B illustrates enlarging and securing the conduit in the channel using an expandable member or balloon 560. The balloon 560 may be radially expanded using fluid (gas or liquid) pressure to deploy the conduit 500. The balloon may have a cylindrical shape (or another shape such as an hourglass shape) when expanded to 1.) expand the center section and 2.) deflect the proximal and distal sections of the conduit such that the conduit is secured to the tissue wall 520. During this deployment step, the tissue wall 520 may distort or bend to some degree but when the delivery tool is removed, the elasticity of the tissue tends to return the tissue wall to its initial shape. Accordingly, the conduits disclosed herein may be deployed either perpendicular to (or non-perpendicular to ) the tissue wall.

Conduit Maintenance

The deployed conduits may be cleared periodically to remove blockages in the conduit's passageway. A wide variety of devices or instruments may clear the passageway. For example, the devices may use heat, laser, electrical energy, ultrasound, radiation, pressure, cutting, etc. to remove the blockage.

Additionally, various conduit designs may have inherent components that are activatable to clear or remove tissue blockages. The components may be actuated based on various stimuli or signals such as temperature, pressure, light, or another process or may be actuated periodically or simply as desired. For example, a conduit may comprise a member that expands when heated. Upon heating the member, the member moves blockages from the passageway of the conduit, allowing air to pass through. The member may be a shape memory material and may be heated by passing a medical device into the lungs and mechanically or electrically connecting with the conduit. Additionally, the conduit may be equipped with a small externally activatable actuator that moves a penetrating member through the conduit's passageway when activated. Alternatively, the conduit may comprise an electrode. When activated, the electrode provides heat or a discharge current that inhibits or ablates scar tissue and tissue overgrowth. Another conduit implant may contain a reservoir that holds a bioactive substance or poison which inhibits tissue overgrowth or build-up. The reservoir has a first shape at one temperature and a second “open” shape at another temperature. Upon heating the reservoir, the substance may be released. For activatable designs including those listed above, activation may also be accomplished by passing electromagnetic waves from an external device through the chest wall. Accordingly, periodic maintenance or activation prevents tissue overgrowth from blocking the conduit's passageway.

The conduits may also include microchips that are remotely powered or controlled. Examples of microchip reservoir devices using wireless transmission of power and data are disclosed in U.S. patent application Ser. No. 2002/0072784.

A medical kit for improving gaseous flow within a diseased lung may include a conduit, a hole-making device, a deployment device and/or a detection device. Examples of such methods and devices are described in U.S. patent application Ser. No. 09/633,651, filed on Aug. 7, 2000; U.S. patent application Ser. Nos. 09/947,144, 09/946,706, and 09/947,126 all filed on Sep. 4, 2001; U.S. patent application Ser. Nos. 10/080,344 and 10/079,605 both filed on Feb. 21, 2002; and U.S. patent application Ser. No. 10/235,240 filed Sep. 4, 2002 each of which is incorporated by reference in its entirety. The kit may further contain a power supply, such as an RF generator, or a Doppler controller which generates and analyzes the signals used in the detection devices. The kit may include these components either singly or in combination.

The kit of the present invention may also contain instructions teaching the use of any device of the present invention, or teaching any of the methods of the present invention. The instructions may actually be physically provided in the kit, or it may be on the covering, e.g., lidstock, of the kit. Furthermore, the kit may also comprise a bronchoscope, or guide-member (such as a guide-wire), or other such device facilitating performance of any of the inventive procedures described herein. All the components of the kit may be provided sterile and in a sterile container such as a pouch or tray. Sterile barriers are desirable to minimize the chances of contamination prior to use.

Creating Incisions and Folds

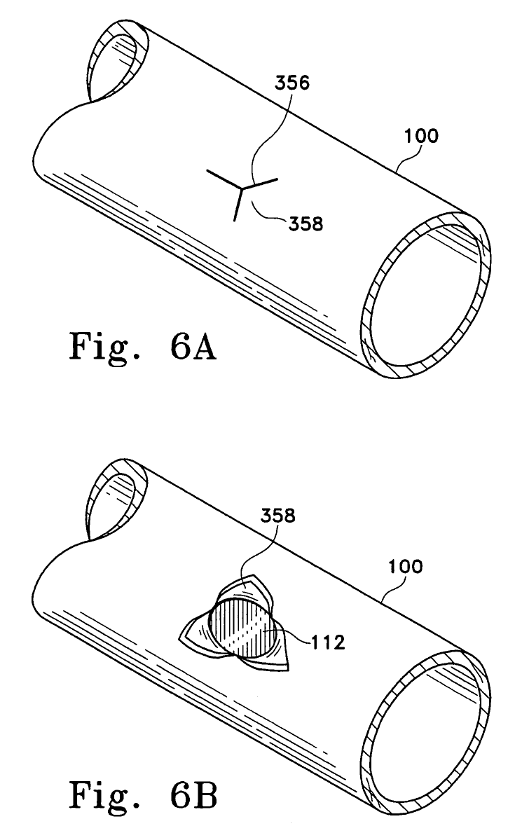

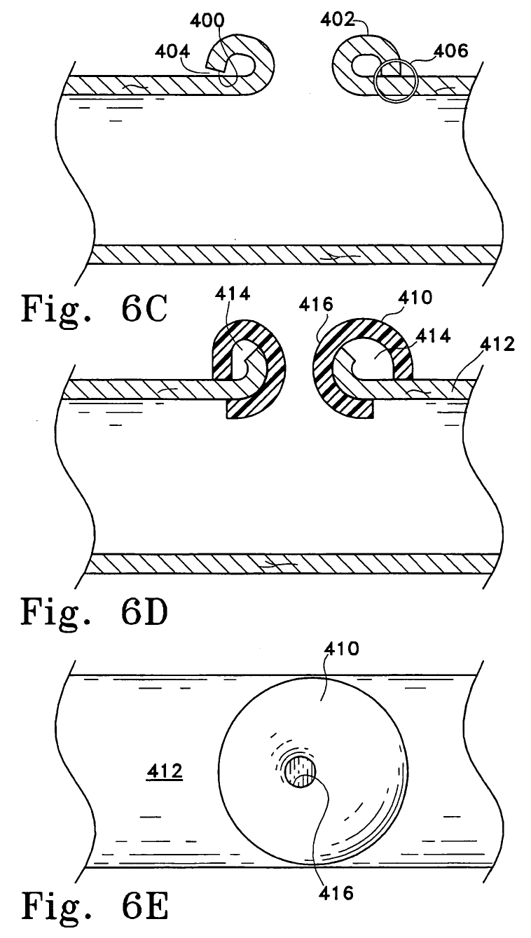

The invention also includes creating a collateral channel by making a single or a series of incisions in an airway wall then folding back (or invaginating) the tissue to form the collateral channel. This procedure allows the surface epithelium which was previously on the inside of the airway wall to cover the walls of the newly formed collateral channel. As discussed herein, promoting growth of the epithelium over the walls of the collateral channel can provide a beneficial healing response. The incision may be created by the use of heat or a mechanical surface. For example, FIG. 6A illustrates a section of an airway 100 having several incisions 356 forming a number of sections 358 of airway wall tissue in the airway 100. FIG. 6B illustrates the sections or flaps 358 of the airway wall folded through the collateral channel 112. Any number of incisions 356 may be made to form any number of sections 358 of airway wall tissue as desired. For example, a plus-shaped incision would result in four sections of tissue that may be folded through a channel. The sections 358 may be affixed with a suture material, an adhesive, or the sections 358 may simply be inserted into surrounding tissue to remain folded through the collateral channel 112.

A cross sectional view of a surgically-created channel is shown in FIG. 6C. In this figure, an edge 400 of a tissue section 402 is folded to contact a portion 404 of tissue not altered by the surgical creation of the channel. The presence of the healthy tissue may signal to terminate (or decrease) the wound healing response so that cell migration, exudation, and other wound-healing phenomena which typically cause the wound (opening) to close are minimized. The sections 402 of tissue may be folded or rolled onto themselves as shown in FIG. 6C using sutures 406 or other biocompatible fastening materials. Also, a conduit as described herein may be deployed in the channel to maintain or bolster its patency. The tissue sections may also be folded distally toward the parenchyma, as shown in FIG. 6C, or proximally, toward the airway, not illustrated. Bioactive agents may be delivered systematically or locally to inhibit tissue overgrowth and or to increase adhesion between the conduit and the channel.

Another configuration to promote folding of the tissue sections is shown in FIG. 6D. In this figure, a conduit 410 is shaped such that it redirects the tissue section as the wound heals. In particular, the tissue wall 412 may be focussed or directed into space 414 defined by the conduit 410. The conduit may be asymmetrical about the tissue wall such that the space 414 is present only on the distal side of the airway wall or the parenchyma tissue side. Also, the conduit may be symmetrical about the airway wall. In this figure, the tissue sections are rolled or folded outwardly rather than inwardly. However, the tissue sections may also be rolled or folded inwardly (i.e., into the airway). Accordingly, space 414 may be provided on the distal portion of the conduit 410. Even if tissue wall 412 continues to grow, its growth will be confined to space 414 such that the collateral opening 416 will not be occluded.

It is also contemplated that adhesives, sealant or medicines may be added to space 414 to maintain the conduit in position, to maintain the tissue wall in a controlled shape, and to inhibit tissue overgrowth. Such materials may be supplied after the conduit is deployed. Also, the materials may be coated (or otherwise impregnated) onto the conduits prior to deployment. The conduit may have yet other shapes and constructs as described herein including various surface coatings, tissue barriers, and other materials which may help prevent the conduit from being ejected and prevent tissue ingrowth.

Conduit-Free Techniques for Maintaining Patency

Although the present invention typically includes an implantable conduit as described above, the present invention is not so limited and the surgically created channels may be maintained without the use of a conduit. For example, a surgically created channel may be maintained by deploying an implant such as a plug, mandrel or sponge for a period of time sufficient to allow the tissue to heal coaxially around the sponge. Once the tissue is healed, the sponge may be removed. Suitable materials for the sponge include those described above and those used in conventional sponges. Also, the sponge may be biodegradable such that after the tissue sets coaxially around the sponge, the sponge disintegrates leaving an open lumen. The sponge may also be loaded with bioactive agents and medicines as described above.

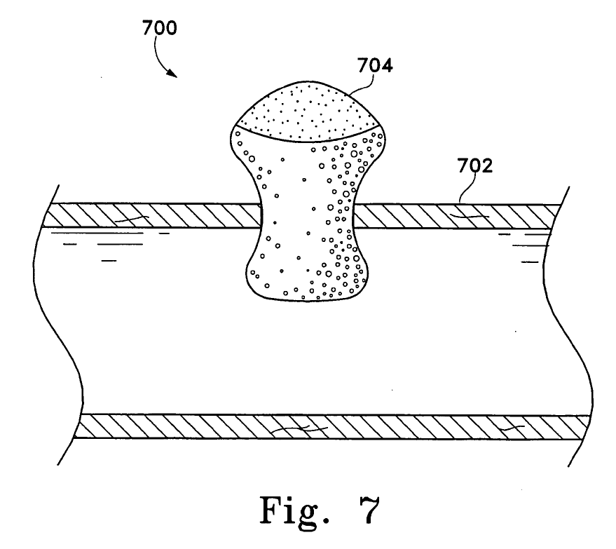

FIG. 7 illustrates an implant 700 deployed in an airway wall 702. The implant may be an open (or closed) cell foam material having a discrete bioactive coating 704. The implant may have an hourglass type shape so that it remains in the opening in the airway wall. Also, the coating may be disposed on a portion of the implant or the entire implant. FIG. 7 depicts an implant having a coating on a distal end of the implant. That is, the coating is disposed on the end that is towards the parenchyma. The implant may be comprised of other materials as disclosed elsewhere in this application.

Tissue grafts such as vein or airway grafts may be fastened in the surgically created channels to provide collateral openings for air to flow through. The tissue grafts may be excised from human and non-human mammals (i.e., allogeneic or xenogeneic). It may be tissue from the same individual (autologous) or another individual. The graftable tissue may be bonded to the airway using, for example, cyanoacrylate. The graft may be inverted, or turned inside out, to facilitate placing the epithelial or endothelial surface of the graft on the outside of the graft.

The channels and conduits described herein may be cleared periodically to remove any blockages. A wide variety of devices or instruments may clear the passageway. Thermal energy, laser, electrical energy, ultrasound, radiation, mechanical cutting, cryogenic energy, etc. are non-limiting examples of means which may clear the passageway.

Additionally, bioactive agents may be deployed locally or systemically to prevent tissue growth from blocking the channels. The bioactive agents may supplement implants or they may be used independent of the implants.

All publications, patent applications, patents, and other references mentioned herein are incorporated by reference in their entirety. To the extent there is a conflict in a meaning of a term, or otherwise, the present application will control. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. It is also contemplated that combinations of the above described embodiments/variations or combinations of the specific aspects of the above described embodiments/variations are within the scope of this disclosure.

1. A conduit for maintaining the patency of a channel created in tissue, said conduit having a low-profile delivery state when the conduit is being delivered to said channel and an expanded deployed state when the conduit is deployed in said channel, said conduit comprising:
a radially expandable center section having a first end and a second end and a passageway extending between said first and second ends, said passageway having an axis;
at least one extension member extending from each of said first end and said second end of said center section, each of said extension members having a fixed end connected to one of said first and second ends of the center section and a movable end such that each of said extension members is capable of being deflected about said fixed end to form an angle with said axis when said conduit is in said deployed state; and
a bioactive substance disposed on at least a portion of a surface of said conduit.

2. The conduit of claim 1, wherein said center section comprises a mesh formed from a plurality of ribs and said center-control segment connects at least one rib to an adjacent rib.

3. The conduit of claim 1, further comprising a tissue barrier coaxially covering said passageway, said tissue barrier forming said exterior surface.

4. The conduit of claim 3, wherein said tissue barrier further covers at least a portion of said extension members.

5. The conduit of claim 4, further comprising at least one visualization feature disposed on a portion of said tissue barrier.

6. The conduit of claim 5, wherein said visualization feature is a stripe circumferentially disposed about at least a portion of said center section.

7. The conduit of claim 2, wherein said bioactive substance is selected from the group consisting of antimetabolites, antithrobotics, anticoagulants, antiplatelet agents, thorombolytics, antiproliferatives, antinflammatories, agents that inhibit hyperplasia, agents that inhibit restenosis, smooth muscle cell inhibitors, growth factors, growth factor inhibitors, cell adhesion inhibitors, cell adhesion promoters, drugs that enhance the formation of healthy neointimal tissue, analgesics, anticonvulsives, antiinfectives, antineoplastics, Histamine 2 antagonists, steroids, non-steroidal antiinflammatories, hormones, immunomodulators, mast cell stabilizers, nucleoside analogues, respiratory agents, antihypertensives, antihistamines, ACE inhibitors, cell growth factors, nerve growth factors, anti-angiogenic agents, angiogenesis inhibitors, tissue irritants, poisons, cytotoxic agents, metals, silver, arsenic, pyrolitic carbon, titanium-nitride-oxide, taxanes, paclitaxel, fibrinogen, collagen, thrombin, phosphorylcholine, heparin, rapamycin, radioactive 188Re and 32P, silver nitrate, dactinomycin, sirolimus, everolimus, Abt-578, tacrolimus, camtophecin, etoposide, vincristine, mitomycin, fluorouracil, and cell adhesion peptide.

8. The conduit of claim 2, further comprising a binding agent disposed on the exterior surface of the conduit such that the bioactive substance adheres to the conduit, at least in part, via the binding agent.

9. The conduit of claim 1, further comprising at least one center-control segment configured to restrict radial expansion of said passageway to a maximum profile.

10. The conduit of claim 1, wherein said conduit is constructed to automatically assume its deployed state.

11. The conduit of claim 1, wherein when said conduit is radially expanded said conduit has an overall length and an inner diameter such that a ratio of the overall length to the inner diameter ranges from 1/6 to 2/1 12. The conduit of claim 11, wherein said ratio ranges from 1/4 and 1/1.

12. The conduit of claim 11, wherein said ratio ranges from 1/4 and 1/1.

13. The conduit of claim 12, wherein said ratio ranges from 1/4 to 1/2.

14. The conduit of claim 11, wherein said exterior surface is a tissue barrier coating coaxially disposed over at least the center section of the conduit.

15. The conduit of claim 14, wherein said tissue barrier coating is a polymeric coating.

16. The conduit of claim 11, wherein said bioactive substance is contained in a polymeric matrix that is configured to gradually release said substance from said matrix.

17. The conduit of claim 1, where the bioactive agent is contained in a polymer matrix, where the polymer matrix is loaded onto the conduit such that the polymer matrix readily detaches from the conduit.

18. A method for improving pulmonary function in an individual comprising:
forming a channel through an airway wall tissue; and
treating the airway tissue such that the channel remains open to allow airflow through the channel into the airway.

19. The method of claim 18, wherein treating the airway tissue comprises inhibiting healing of the airway wall tissue.

20. The method of claim 19, wherein inhibiting comprises delivering a medicine to the channel.

21. The method of claim 19, wherein inhibiting comprises delivering a medical device to the channel that at least physically prevents the channel from closing.

22. The method of claim 21, comprising delivering a substance that does not induce tissue encapsulation of the medical device.

23. The method of any of claims 19, comprising delivering energy to the channel to inhibit healing of the airway.

24. The method of claim 18, where treating the airway tissue comprises preventing ingrowth of tissue into the channel.

25. The method of claim 24, wherein treating the airway tissue comprises impeding the wound healing process of lung tissue such that the lung tissue cannot heal and the channel remains patent.

26. The method of claim 24, wherein treating the airway tissue comprises accelerating the wound healing process such that the channel remains patent.

27. The method of claim 26, wherein the step of accelerating the wound healing process comprises increasing the growth of epithelial cells.

28. The method of claim 24, treating the airway tissue comprises inserting a conduit in said channel.

29. The method of claim 28, further comprising treating the lung tissue with a bioactive substance.

30. The method of claim 29, wherein said treating the lung tissue is performed by supplying said bioactive substance on a surface of said conduit.

31. The method of claim 30, wherein said conduit includes a tissue barrier coaxially surrounding at least a center section of said conduit and said bioactive substance is disposed on said tissue barrier.

32. The method of claim 31, wherein said bioactive substance is selected from the group consisting of antimetabolites, antithrobotics, anticoagulants, antiplatelet agents, thorombolytics, antiproliferatives, antinflammatories, agents that inhibit hyperplasia, agents that inhibit restenosis, smooth muscle cell inhibitors, growth factors, growth factor inhibitors, cell adhesion inhibitors, cell adhesion promoters, drugs that enhance the formation of healthy neointimal tissue, analgesics, anticonvulsives, antiinfectives, antineoplastics, Histamine 2 antagonists, steroids, non-steroidal antiinflammatories, hormones, immunomodulators, mast cell stabilizers, nucleoside analogues, respiratory agents, antihypertensives, antihistamines, ACE inhibitors, cell growth factors, nerve growth factors, anti-angiogenic agents, angiogenesis inhibitors, tissue irritants, poisons, cytotoxic agents, metals, silver, arsenic, pyrolitic carbon, titanium-nitride-oxide, taxanes paclitaxel, fibrinogen, collagen, thrombin, phosphorylcholine, heparin, rapamycin, radioactive 188Re and 32P, silver nitrate, dactinomycin, sirolimus, everolimus, Abt-578, tacrolimus, camtophecin, etoposide, vincristine, mitomycin, fluorouracil, and cell adhesion peptide.

33. The method of claim 30, wherein said conduit includes a tissue barrier coaxially surrounding at least a center section of said conduit and said tissue barrier is at least partially formed of said bioactive substance.

34. The method of claim 33, wherein said bioactive substance is selected from the group consisting of antimetabolites, antithrobotics, anticoagulants, antiplatelet agents, thorombolytics, antiproliferatives, antinflammatories, agents that inhibit hyperplasia, agents that inhibit restenosis, smooth muscle cell inhibitors, growth factors, growth factor inhibitors, cell adhesion inhibitors, cell adhesion promoters, drugs that enhance the formation of healthy neointimal tissue, analgesics, anticonvulsives, antiinfectives, antineoplastics, Histamine 2 antagonists, steroids, non-steroidal antiinflammatories, hormones, immunomodulators, mast cell stabilizers, nucleoside analogues, respiratory agents, antihypertensives, antihistamines, ACE inhibitors, cell growth factors, nerve growth factors, anti-angiogenic agents, angiogenesis inhibitors, tissue irritants, poisons, cytotoxic agents, metals, silver, arsenic, pyrolitic carbon, titanium-nitride-oxide, taxanes, paclitaxel, fibrinogen, collagen, thrombin, phosphorylcholine, heparin, rapamycin, radioactive 188Re and 32P, silver nitrate, dactinomycin, sirolimus, everolimus, mitomycin, Abt-578, tacrolimus, camtophecin, etoposide, vincristine, fluorouracil, and cell adhesion peptide.

35. The method of claim 18, wherein treating airway tissue comprises inhibiting closure of the channel.

36. The method of claim 35, wherein treating the airway tissue comprises irradiating at least a portion of the channel.

37. The method of claim 35, wherein treating the airway tissue comprises deployment of a medical device into said channel.

38. The method of claim 37 further comprising delivering a bioactive substance into or around the channel.

39. The method of claim 38, where the bioactive substance is located within a polymer carrier.

40. The method of claim 38 wherein the bioactive substance is delivered locally.

41. The method of claim 38, wherein the bioactive substance is delivered systemically.

42. The method of claim 37, wherein the bioactive substance is disposed on an exterior surface of the device.

43. The method of claim 37, wherein the bioactive substance is contained in a reservoir of said medical device.

44. The method of claim 37, wherein bioactive substance is mixed with a biodegradable compound and disposed on an exterior surface of said medical device, said biodegradable compound different from said medical agent.

45. The method of claim 37, wherein bioactive substance is covered by an outer biodegradable substance.

46. The method of claim 37, wherein the medical device is a conduit.

47. The method of claim 38, wherein the bioactive substance is selected from the group consisting tissue growth inhibitors, tissue growth enhancers, anti-microbial agents, anti-inflammatory agents, biological reaction inhibitors, immune-response inhibitors, antimetabolites, steroids, metals, and anti-infection agents.

48. The method of claim 38, wherein the bioactive substance is selected from the group consisting of pyrolitic carbon, titanium-nitride-oxide, taxanes, paclitaxel, fibrinogen, collagen, thrombin, phosphorylcholine, heparin, rapamycin, radioactive 188Re and 32P, silver nitrate, dactinomycin, sirolimus, everolimus, mitomycin, fluorouracil, Abt-578, tacrolimus, camtophecin, etoposide, vincristine, or cell adhesion peptide.

49. The method of claim 35, comprising deploying a vessel graft in said channel, said vessel graft having a passageway for air to flow through.

50. The method of claim 37, wherein said medical device is a sponge.

51. The method of claim 50, further comprising removing said sponge.

52. The method of claim 50, wherein said sponge is biodegradable.

53. The method of claim 50, wherein the sponge comprises a medical agent.

54. The method of claim 35, wherein said inhibiting closure includes systemically delivering a medicine.

55. The method of claim 54, wherein said systemically delivering a medicine is performed via any one of the following ways of ingestion, inhalation, injection, and absorption.

56. The method of claim 55, wherein said channel remains device free.

57. The method of claim 35, wherein inhibiting closure includes applying thermal energy to at least a portion of the channel.

58. The method of claim 49, wherein said graft has been inverted prior to said deploying step.
